(19) United States
(12) Patent Application Publication
(10) Pub. No.: US 2026/0124120 A1
(43) Pub. Date: May 7, 2026

(54) STABILIZED AQUEOUS COMPOSITIONS CONTAINING HYDROXYAPTITE AND FREE IONS FOR IN VIVO REMINERALIZATION OF CALCIFIED TISSUE & THEIR METHODS OF USE

(71) Applicant: INTER-MED, INC., Racine, WI (US)

(72) Inventors: Alexander D. JOHNSON, Racine, WI (US); Khongchee Xiong, Racine, WI (US)

(21) Appl. No.: 19/440,333

(22) Filed: Jan. 5, 2026

Related U.S. Application Data
(63) Continuation of application No. PCT/US2025/035955, filed on Jun. 30, 2025.
(60) Provisional application No. 63/666,880, filed on Jul. 2, 2024.

(51) Int. Cl. A61K 6/20; A61K 6/17; A61K 6/69

(52) U.S. Cl. CPC A61K 6/20; A61K 6/17; A61K 6/69

(57) ABSTRACT

Aqueous-based compositions for remineralizing dentinal tissues in vivo are disclosed to arrest demineralization and early-stage caries, treat dentin/tooth sensitivity, fix white spot lesions, and strengthen dentinal hard tissues (enamel, dentin, cementum), among others. Methods utilizing the remineralizing composition are also disclosed.

### CROSS-REFERENCE TO RELATED APPLICATIONS

This application is a continuation of International Application No. PCT/US2025/035955, filed Jun. 30, 2025, which claims the benefit of U.S. Provisional Application No. 63/666,880, filed Jul. 2, 2024; the entire contents of those applications are hereby incorporated by reference herein.

### BACKGROUND OF INVENTION

Human teeth are living structures comprised of both hard and soft tissues. The hard tissue consists of enamel (the hard exterior surface tissue of teeth), dentin (immediately below enamel and helps support enamel), and cementum (a specialized hard tissue that covers the root of a tooth). The dental pulp, generally referred to as soft tissue, primarily resides within the pulp chamber. Pulp tissue consists of many types of cells and connective tissue but also includes blood vessels and nerves that enter the tooth from the apical foramen, which is a hole at the apex of a root canal.

Demineralization is a process where minerals are gradually lost from dentinal hard tissues due to factors including oral bacteria, acidic foods, and inadequate saliva. This demineralization can lead to increased tooth sensitivity, visible white spots, and ultimately the development of dental caries, which necessitates dental treatment to restore the destroyed or decayed tooth structure.

In an effort to arrest demineralization before it progresses, several treatment modalities exist. The most common treatment is the application of a fluoride-containing material, such as a fluoride varnish or fluoride gel/paste, to the treatment site which helps strengthen the enamel and dentin through the formation of fluorapatite that is more resistant to acidic attack. However, excessive fluoride application can lead to unsightly fluorosis spots on teeth, and in extreme cases, fluorosis can weaken the tooth. An alternative material, called MI Paste, utilizes a milk-derived protein to help bind, adhere and release calcium, phosphate and fluoride to the tooth surface to treat sensitivity and demineralization. MI Paste exhibits limitations and potential disadvantages related to excessive fluoride application while also being a potential allergen due to the inclusion of a milk-derived protein. Furthermore, the inclusion of calcium and fluoride within the same chemistry may result in the precipitation of calcium fluoride, which is insoluble in aqueous media and renders calcium and fluoride ions useless for remineralization.

U.S. Pat. No. 9,724,542 describes a composition containing remineralizing ingredients, including fluoride, in an aqueous-free emulsion that forms mucoadhesive gels when in contact with tooth surfaces and saliva. U.S. Pat. No. 10,279,203 describes a composition containing remineralizing ingredients that rely on delivery via pores contained with silicon dioxide microparticles.

Therefore, there is a need in the art for methods and compositions to remineralize dentinal hard tissues in vivo that is appreciably fluoride free and does not include the use of animal or synthetically derived peptides or proteins.

### BRIEF SUMMARY

The present disclosure provides aqueous-based compositions for remineralizing dentinal tissues in vivo. The remineralizing compositions disclosed herein are useful, without limitation, to arrest demineralization and early-stage caries, treat dentin/tooth sensitivity, fix white spot lesions, and strengthen dentinal hard tissues (enamel, dentin, cementum), among others. Although the disclosure is generally geared towards dental use, one skilled in the art can appreciate how the disclosed inventions may be applicable to other medical and orthopedic applications where remineralization of bone or other tissues is clinically advantageous. The present disclosure also teaches methods utilizing the remineralizing composition. In yet another aspect, a product by process to create the remineralizing composition is disclosed. In an even further aspect, kits containing the composition are disclosed.

### BRIEF DESCRIPTION OF THE DRAWINGS

FIG. 1 shows calcium phosphate precipitation and remineralization of hydroxyapatite powder overtime when exposed to an embodiment of the application.

FIG. 2 shows in situ bioactive material formation within the remineralization composition (Formula A) versus different pHs.

FIG. 3 shows laser microscope images at 50× of dentin tubules post seven-day treatment protocol. Nearly all of tubules are open within the negative control image which corresponds to a grading score of “0”. Conversely, ˜100% of the tubules are closed within the Formula A image which corresponds to a grading score of “4” and illustrates how an embodiment of the application effectively remineralizes dentin and occludes dentinal tubules, thereby eliminating patient sensitivity. Approximately 25% of the tubules appear closed within the SuperSeal image which corresponds to a grading score of “1”.

FIG. 4 shows bioactive material formation (apatites and apatite precursors) versus remineralizing solution pH. More apatites were formed at pHs between 7-8 whereas more acidic pHs led to more apatite precursor formation.

FIG. 5 shows bioactive material formation (apatites and apatite precursors) versus manufacturing method of the remineralizing solution. More apatites were formed utilizing manufacturing method #2 versus manufacturing method #1.

FIG. 6 shows total bioactive material formation (apatites and apatite precursors) versus manufacturing method of the remineralizing solution. More bioactive materials were formed utilizing manufacturing method #2 versus manufacturing method #1.

FIG. 7 shows Dentin Hypersensitivity Pain Level using Schiff Air Index Scoring.

FIG. 8 shows Dentin Hypersensitivity Pain Level using Patient Self Scoring Results.

FIG. 9 shows Treatment of a white spot lesion (WSL).

### DESCRIPTION OF THE INVENTION

The following paragraphs define in more detail the embodiments of the invention described herein. The following embodiments are not meant to limit the invention or narrow the scope thereof, as it will be readily apparent to one of ordinary skill in the art that suitable modifications and adaptations may be made without departing from the scope of the invention, embodiments, or specific aspects described herein.

The remineralizing composition comprises at least two inorganic salts, at least one bioactive material, at least one buffering agent, at least one pH modifier, and at least one solvent wherein at least a first inorganic salt yields free calcium (Ca²⁺) ions in water, and wherein at least a second inorganic salt yields free phosphate ions in water, wherein the remineralizing composition is substantially fluoride-free, and wherein the remineralizing composition does not contain animal derived ingredients. In certain embodiments, the remineralizing composition comprises at least three inorganic salts, at least one bioactive material, at least one buffering agent, at least one pH modifier, and at least one solvent wherein at least a first inorganic salt yields free calcium (Ca²⁺) ions in water, wherein at least a second inorganic salt yields free phosphate ions in water, and wherein at least a third inorganic salt does not contain calcium or phosphate, wherein the remineralizing composition is substantially fluoride-free, and wherein the remineralizing composition does not contain animal derived ingredients. The ratio between the calcium containing inorganic salt and phosphate containing inorganic salt is critical to the composition, and may be any ratio between 5:1 and 1:5 but is preferably between 2:1 and 1:2, and more preferably about 2:1. Ideally, the molar ratio of calcium ions and phosphate ions is preferably between 1:1 and 2:1, and is more preferably between 1.5:1 and 2:1, and most preferably about 1.7:1. Optionally, the composition may include one or more of the following: at least one thickener, at least one flavorant, at least one sweetener, at least one surfactant, at least one hydrotrope, at least one colorant, at least one antimicrobial agent, at least one preservative, at least one prebiotic, at least one antioxidant, or at least one probiotic. Generally, all ingredients should exhibit transient biocompatibility for safety and toxicity considerations. It may be possible that a single ingredient satisfies one or more of these optional ingredient classifications, for example, xylitol is a sweetener and antimicrobial agent. Additionally, although the remineralizing composition is preferably fluoride-free, it may be advantageous in certain instances to include a small amount of fluoride (preferably less than 5000 ppm, more preferably less than 500 ppm, even more preferably less than 100 ppm, and most preferably less than 10 ppm). Thus, a substantially fluoride-free composition is one in which the amount of fluoride is present at less than 5000 ppm.

In certain embodiments, the remineralizing composition includes at least three inorganic salts, wherein at least a first inorganic salt yields free calcium (Ca²⁺) ions in water, wherein at least a second inorganic salt yields free phosphate ions in water, and wherein at least a third inorganic salt does not contain calcium or phosphate. Depending on the pH, the free phosphate may be orthophosphate (also simply referred to as phosphate, PO₄³⁻), dihydrogen phosphate (H₂PO₄⁻), or hydrogen phosphate (HPO₄²⁻). The inorganic salts are preferably appreciably water-soluble, however, in other embodiments, the inorganic salts may only be slightly water soluble, which may provide a reservoir for ion release as free aqueous ions are consumed in remineralization of dentinal hard tissues. In other words, based on Le Chatlier's principle, as aqueous free ions are consumed during remineralization, the chemical equilibrium of the slightly soluble inorganic salt will shift to produce more aqueous free ions which then can be used for further remineralization. Accordingly, the slightly soluble inorganic salt acts as a reservoir to replace aqueous free ions that are consumed during remineralization. In certain embodiments, the inorganic salts may be salt hydrates (e.g., calcium chloride dihydrate, magnesium chloride hexahydrate, etc.). The remineralizing composition may be generally designed to mimic body fluid but includes at least one inorganic salt that yields excess free calcium (Ca²⁺) ions in water and includes at least one inorganic salt that yields excess free phosphate ions in water. An exemplary formula that accomplishes this goal could contain various salts (inorganic and organic) to yield the following aqueous ion concentrations: sodium 120-160 mM, potassium 3-7 mM, magnesium 1-2 mM, calcium 2-5,000 mM, chloride 50-200 mM, bicarbonate 4-50 mM, phosphate 0.5-5,000 mM, and sulfate. By mimicking body fluid, the remineralizing composition is more effective at remineralizing tooth structure than other competitive products. Generally, the inorganic salts comprising the remineralizing composition are at a weight/weight concentration (w/w %) between 0.01%-5%, and preferably between 0.01%-2%. Furthermore, the inorganic salts are chosen and balanced accordingly such that the osmolarity of the remineralizing composition is isotonic (270-310 mOsm/L) to slightly hypertonic (310-1000 mOsm/L).

It may be advantageous to include at least one bioactive material or substate that helps accelerate and promote remineralization in vivo. Example bioactive materials include, but are not limited to: bioglass, tricalcium phosphate, apatites (including hydroxyapatite (HAp), carbonate substituted hydroxyapatite (CHAp), chloride substituted hydroxyapatite (ClHAp), and other apatites), monetite, brushite, tetracalcium phosphate, dicalcium phosphate, monocalcium phosphate, other calcium phosphate salts, among others and combinations thereof. In certain embodiments, the at least one bioactive material may comprise apatites (including hydroxyapatite (HAp), carbonate substituted hydroxyapatite (CHAp), chloride substituted hydroxyapatite (ClHAp), and other apatites), apatite precursors (including monetite, brushite, tricalcium phosphate, tetracalcium phosphate, dicalcium phosphate, monocalcium phosphate, and other calcium phosphate salts) and combinations thereof. ClHAp may be preferred in certain instances as it is also considered an antimicrobial agent. It may be preferred to tailor the use of various apatites for different remineralization rates in certain clinical applications. For example, CHAp and ClHAp will release more biologically active ions (calcium and phosphate) quicker than HAp. Therefore, in certain embodiments it may be useful to have a combination of various apatites in the composition to facilitate different ion release profiles and remineralization.

In certain instances, the bioactive material is added directly to the composition during manufacturing (i.e. as its own ingredient), while in other preferred instances, the bioactive material is created within the formulation during manufacturing, because of manufacturing, or after manufacturing. For example, depending on the concentration of free calcium ions and free phosphate ions, along with the composition's pH, the in-situ formation of calcium phosphate salts, including apatites (e.g. hydroxyapatite (HAp), carbonate substituted hydroxyapatite (CHAp), chloride substituted hydroxyapatite (ClHAp), or other apatites), and apatite precursors (e.g. monetite and brushite) will form within the remineralizing composition during manufacturing or shortly after manufacturing is complete; this in-situ formation of apatites and apatite precursors may result in a decrease in pH of the remineralizing composition so the composition shall be designed to maintain a pH of at least 4.5 and more preferably at least 5.5. In certain embodiments, this process results in the remineralizing composition containing stabilized and suspended apatite particles (for example, hydroxyapatite particles) along with apatite precursors (e.g. calcium and phosphate salts), and free calcium ions and phosphate ions in equilibrium. This is preferred as the apatite particles, hydroxyapatite precursors, and free ions (calcium and phosphate) provide significant remineralization benefits when applied in vivo. Depending on the ion balance within the remineralizing composition, various other apatites and salt precipitates may be formed advantageous in situ within the remineralizing composition. In certain embodiments, the in situ formed bioactive material is comprised of calcium phosphate apatite salts, which consist of preferably at least 30% apatite, more preferably at least 50% apatite, and most preferably at least 65% apatite. In certain embodiments, the formed apatite is comprised of HAP, CHAp, ClHAp, amongst others and combinations thereof. In other embodiments, the in situ formed bioactive material is comprised of apatite precursors (including calcium phosphate salts), which consist of preferably at least 30% apatite precursors, more preferably at least 50% apatite precursors, and most preferably at least 65% apatite precursors. In certain embodiments, the formed apatite precursor is comprised of monetite, brushite, tricalcium phosphate, tetracalcium phosphate, dicalcium phosphate, monocalcium phosphate, amongst others and combinations thereof. It may be advantageous to utilize a specific ratio between apatite and apatite precursors within the composition, which may be any ratio between 20:1 and 1:20 but is preferably between 1:1 and 10:1. Nevertheless, whether the bioactive material is added as an ingredient to the remineralizing composition during manufacturing or is created in situ as a result of manufacturing, the bioactive material particles within the remineralizing composition are preferably less than 50 microns in size, more preferably less than 20 microns in size, even more preferably less than 5 microns in size, and most preferably less than 1 micron in size (i.e. nanosized).

At least one buffering agent is included within the remineralizing composition at a weight/weight concentration (w/w %) between 0.1%-5%, and preferably between 0.1%-1%. Example buffering agents can include, but are not limited to: MES, Bis-Tris, ADA, ACES, PIPES, MOPSO, Bis-Tris propane, BES, MOPS, TES, HEPES, DIPSO, MOBS, TAPSO, Tris or Trizma, HEPPSO, POPSO, TEA, EPPS, Tricine, Gly-Gly, Bicine, HEPBS, TAPS, and AMPD, amongst others and combinations thereof. The remineralizing composition has a pH preferably between 3-9, more preferably between 4-8, and most preferably between 4.5-7.5, so the buffer shall be chosen for optimized performance within the target pH ranges. The inclusion of at least one pH modifier (e.g., hydrochloric acid) is utilized to ensure the target pH is obtained. Alkaline pHs (>9) shall be avoided to minimize the undesired precipitation of insoluble metal hydroxides. In certain embodiments, it is clinically advantageous for the remineralization composition to have a slightly acidic pH (≤6.5) to slightly etch the tooth surface for improved adhesion.

Sugar alcohols (i.e. a type of sweetener) or flavorants may be used as a palletizer within the remineralizing composition at a weight/weight concentration (w/w %) between 0.1% and 5%, and in some embodiments, between 1% and 2%. Anti-microbial properties may be a reason to include sugar alcohol due to the minimal caloric value it supplies to bacteria, thereby preventing multiplication of colonies that otherwise may use sucrose or other sweeteners as a food source. Suitable sugar alcohols may include, but are not limited to, sorbitol, mannitol, maltitol, isomalt, erythritol, xylitol, and the like. Other sweeteners may include, but are not limited to, allulose, acesulfame potassium, aspartame, cyclamate, mogrosides, saccharin, stevia, and sucralose, amongst others and in combinations thereof. Alternative flavorant agents can include but are not limited to methyl salicylate (wintergreen), menthol, spearmint, peppermint, lavender, lemon, citrus, bubblegum, mango, melon, strawberry, apple, cherry, caramel, and cinnamon, amongst others and in combinations thereof.

The remineralizing composition may also include surfactants to enhance wetting and spreading across the target treatment area. Examples of suitable surfactants (also referred to as wetting agents) include alkyldiphenyloxide disulfonates, alkyl aryl sulfonates, sodium dodecylbenzene sulfonate, alkyl sulfates, alcohol ethoxylates, polyoxyethylene glycol octylphenol ethers, polyoxyethylene glycol alkylphenol ethers, polyoxyethylene glycol sorbitan alkyl esters, sorbitan alkyl esters, copolymers of polyethylene glycol and/or propylene glycol, poloxamers, sodium sterates, sodium lauryl ether sulfates, linear alkylbenzene sulfonates, amine oxides, betaines, sodium lauryl sulfate, polysorbates, lecithin, and disulfonates, fluorosurfactants, among others and in combinations thereof. In some embodiments, the surfactants are nonionic, cationic or zwitterionic, although certain anionic surfactants can be employed. The surfactant is preferably at a weight/weight concentration (% w/w) between 0.01%-20%, more preferably between 0.1%-10%, and most preferably between 0.5%-10%. In certain embodiments, the surface tension of the remineralizing composition is preferably less than 100 dynes/cm, more preferably less than 60 dynes/cm, and most preferably less than 40 dynes/cm.

In certain embodiments, the remineralizing composition comprises at least one calcium chelator, at least one phosphate sequestrant, or combinations thereof. The inclusion of these materials may be advantageous to chelate free aqueous calcium ions or sequester free phosphate ions during manufacturing and storage, which then may provide a source of calcium ions and phosphate ions, respectively, once the remineralizing composition is applied for clinical use in vivo. In other words, the chelated calcium or sequestered phosphate offer a reservoir of bioactive ions (e.g. calcium and phosphate) that can be released in vivo for remineralizing of dentinal hard tissues. Exemplary calcium chelators include, but are not limited to, 1-hydroxyethylidene-1,1-diphosphonic acid (HEDP), ethylenediaminetetraacetic acid (EDTA), phytic acid (PA), sodium hexametaphosphate (SH), N-(phosphonomethyl) iminodiacetic acid (PMIDA), poly (4-styrenesulfonic acid maleic acid) sodium salt (PSS), 2-phosphonobutane-1,2,4-tricarboxylic acid (PBTC), and polyacrylic acid (PAA), among others. Exemplary phosphate sequestrants include, but are not limited to, lanthanum salts, and sevelamer salts, among others.

Depending on the application, the remineralizing composition may have different consistencies. For example, the remineralizing composition may be a liquid for use as an oral rinse; however, thickening agents (i.e. rheology modifiers) may be added to the composition to increase the composition's viscosity to a gel-like consistency for use with dental appliances (e.g. trays) or toothbrushes. In some embodiments the remineralizing composition additionally comprises a rheology modifier that may also optionally serve as a thickener to improve the ability to control the flow of the composition during application. A preferred example are silicon dioxides (for example, fumed silica), preferably at a weight/weight concentration (% w/w) between 0.1%-10%. Another preferred example is poloxamer (for example, poloxamer 407 or poloxamer 188), preferably at a weight/weight concentration (% w/w) between 5%-25%. Poloxamers, specifically poloxamer 407 and poloxamer 188, are preferred in certain embodiments due to their thermoreversible effects which may help promote tissue penetration and ion release of the remineralizing composition in a more liquid state (i.e. less viscous state) prior to poloxamer-induced gelation (i.e. increased viscous state). Furthermore, poloxamers, specifically poloxamer 407 and poloxamer 188, exhibit mucoadhesive or bioadhesive properties that help them cling to tissue more effectively than other thickening agents.

Alternative rheology modifiers (i.e. thickeners) may include but are not limited to: polyvinyl alcohols, polyvinylpyrrolidone, polyacrylic acid, xanthan gum, guar gum, gelatin, agar, ethanol, chitosan, chitosan derivatives, cellulose, cellulose-derivatives, hydroxyethyl cellulose, alginate, other water-miscible solvents, and combinations thereof. If a thinner gel formulation is desired, the thickener is absent or preferably between 1%-10%; however, if a thicker gel formulation is desired, the thickener is preferably between 5%-25%. In some instances, it is preferred if the rheology modifier introduces thixotropic properties to the composition which may promote easier administration as the product thins out during shear stresses induced by administration. Furthermore, thixotropic properties would allow the composition to thin out and become more flowable as it is being applied to the teeth thereby further improving contact of the remineralizing agents with the tooth surfaces. Additional thickeners may be added depending on the consistency of the lubricant composition desired for clinical use. In certain aspects, the composition may be non-Newtonian or pseudoplastic. The disclosed remineralizing composition may have a viscosity of 1-10,000,000 mPa·s, of 10-2,000,000 mPa·s, or of 100-100,000 mPa·s.

Hydrotropes can also be added to surfactants for formulation stability and to reduce phase separation. Certain hydrotropes that can be used in the remineralizing solution include, but are not limited to, sodium xylene sulfonate (SXS), ethylhexyl sulfonate (EHS), and sodium cumene sulfonate (SCS), among others. Furthermore, lubricants may be added to the remineralizing composition. Suitable lubricants can include, but are not limited to, propylene glycol, glycerin, and polyethylene glycol, amongst others. In further embodiments, the remineralizing composition comprises a colorant, pigment or dye that provides visual contrast between the remineralizing composition and the target treatment site. The colorant, pigment, or dye should be chemically stable with the other cleaning solution constituents and not result in any chemical instability (i.e. precipitation formation, etc.). Non-limiting examples of suitable dyes could include any FD&C color dye (particularly Blue No. 1, Blue No. 2, Green No. 3, Orange B, Red No. 2, Red No. 3, Red No. 40, Yellow No. 5, and Yellow No. 6), methylene blue, beta-carotene, or combinations thereof. Nevertheless, regardless of the colorant used, the remineralizing composition should be non-staining. In certain instances, the colorant, pigment, or dye may be ion reactive or ion responsive, meaning the colorant, pigment or dye may exhibit different visual properties depending on the concentration of ions within the composition. For example, this ion reactivity or responsiveness may be useful during in vivo application of the composition to identify areas of demineralization and/or to note when treatment is complete (i.e. a loss of color as free ions in the composition have remineralized dentin tissue).

Antimicrobial agents or preservatives may optionally be added to the remineralizing composition. Suitable antimicrobial agents include elemental iodine (I₂), hypochlorous acid, chlorhexidine, hydrogen peroxide, glutaraldehyde, amongst others. In certain embodiments, the remineralizing composition preferably provides at least a log 3 antimicrobial impact within one minute of use, but more preferably provides at least a log 7 antimicrobial impact within one minute of use. In preferred embodiments, elemental iodine (I₂) is added to the remineralizing composition (as I₂ simultaneously exhibits debriding and antimicrobial properties) at a preferred concentration between 10 parts per million (ppm)-1,000 ppm, a more preferred concentration between 100 ppm-5,000 ppm, and a most preferred concentration between 300 ppm-3,000 ppm. In such a composition, an acidic pH less than 6 is preferred for I₂ stability and efficacy. Furthermore, the concentration of I₂ shall be selected such that brown staining of the dentin does not occur during or after use which would not be aesthetically pleasing in the mouth. In other words, in certain embodiments the remineralizing composition is non-staining. In other embodiments, hydrogen peroxide is added to the composition at a preferable concentration between 0.25%-3%, or at a more preferable concentration between 1.5%-2.5%. In such a composition, a slightly acidic pH less than 6 is preferred for H₂O₂ stability and efficacy. In other embodiments, at least one collagen crosslinker is added to the remineralization composition to exhibit collagen cross linking at a preferable concentration between 0.005%-5%, a more preferable concentration between 0.05%-3.0%, or at a most preferred concentration between 0.5%-2.0%, In such a composition, remineralization on dentin structure is stabilized when collagen cross linking occurs allowing for higher ion uptake and bonding. The at least one collagen crosslinker can include, but is not limited to glutaraldehyde, polyphenols, catechols, carbodiimides (for example, EDC (1-ethyl-3-(3-dimethylaminopropyl) carbodiimide) in conjunction with NHS (N-hydroxysuccinimide)), riboflavin, grape seed extract, genipin, amongst others. In certain instances, the at least one collagen crosslinker also exhibits antimicrobial properties. Preservatives may include, but are not limited to sodium benzoate, citric acid, potassium sorbate, ascorbic acid, parabens (including methylparaben and propylparaben), amongst others and combinations thereof. In certain instances, at least one preservative is added to the composition at a concentration of preferably 10 ppm-10,000 ppm, more preferably 100 ppm-2,500 ppm, and most preferably 400 ppm-1,000 ppm. In certain instances, at least one paraben is added to the composition at a concentration of preferably 10 ppm-1,000 ppm, more preferably 20 ppm-750 ppm, and most preferably 50 ppm-600 ppm.

Optionally, at least one antioxidant may be added to the remineralizing composition to promote oral health and healing of damaged tissue. Antioxidants may include, but are not limited to vitamin C (ascorbic acid), vitamin E, vitamin A, tocopherols, tocotrienols, ferulic acid, phloretin, lycopene, glutathione, amongst others. In certain embodiments, the at least one antioxidant is included at a weight/weight percentage preferably between 0.01%-3.0%, more preferably between 0.1%-2.0%, and most preferably between 0.25%-1.0%.

In further aspects of the disclosure, the remineralizing composition provides a suitable shelf life of about 6-48 months when stored at room temperature without displaying characteristics of chemical instability or significant decreased clinical efficacy. An example of chemical instability would be significant phase separation, during storage that renders the composition less effective or useless. Additionally, in certain embodiments the remineralizing composition passes USP 51 antimicrobial effectiveness testing (AET) and microbial challenge. Specifically, when challenged with P. aeruginosa (ATCC 9027), S. aureus (ATCC 6538), and E. coli (ATCC 8739); C. albicans (ATCC 10231), A. brasiliensis (ATCC 16404), B. cenocepacia (ATCC BAA-245), B. cepacian (ATCC 25416), and B. multivorans (ATCC BAA-247) the remineralizing composition exhibited a microbial log 10 reduction between 4.19 and 4.87 throughout 28 days of testing following USP 51 testing. Accordingly, in certain embodiments the remineralizing solution exhibits a microbial log 10 reduction of preferably of at least 2, more preferably at least 3 and most preferably at least 4.

In certain aspects, the remineralizing composition may be packaged in various syringes or containers sized between 0.1 mL-5000 mL, and in some embodiments, tubes sized between 10 mL-500 mL. In some embodiments of the invention, it may be beneficial to have the remineralizing composition stored in separate containers (e.g. two separate bottles, two separate tubes, dual barrel syringe, etc.) as two distinct components, which the user may mix immediately prior to use.

The packaging of the remineralizing composition must be compatible for long-term storage (months to years). Satisfactory plastic resins for the packaging material may include, but are not limited to, polypropylene, polycarbonate, polyethylene, styrene acrylonitrile, methyl methacrylate-acrylonitrile-butadiene-styrene, ethylene vinyl alcohol copolymer, poly-cyclohexylenedimethylene terephthalate glycol, among others and combinations thereof (for example a five layer tube).

In certain aspects, the remineralizing composition is provided as an item within a kit. In some embodiments, the kit may comprise any one or more of the following components: application tips, application brushes, mixing tips, mixing vessels, empty syringes, an instructions for use, mixing wells or other single use vessels, a dental etchant or etchants, a dental adhesive or adhesives, a dental primer or primers, a dental composite or composites, a dental cement or cements, an endodontic irrigant or irrigants, an endodontic sealer or sealers, and endodontic file or files, among other common dental and endodontic products.

The method of using the remineralizing composition in vivo typically includes introducing the remineralizing composition to the internal or exterior surfaces of a tooth or teeth. The application of the remineralizing composition can vary depending on the intended use of the material and may be applied to wet or dry tooth surfaces. For example, the remineralizing composition in a liquid form may be use as an oral rinse (which would require shaking of the container prior to application to suspend the bioactive material), whereas the remineralizing composition in gel form may be applied intraorally through the use of a toothbrush, applicator brush, dental appliance, or dental tray (e.g. fluoride tray, impression tray, or PerioTray™). Treatment can be targeted to a specific area within the oral cavity (e.g. a specific tooth, implant, quadrant, etc.) or the entire mouth can be targeted. The amount of remineralizing composition introduced intraorally can vary between 0.1 mL-30 mL or more. Additionally, the duration of contact between the remineralizing composition and dental hard tissue can vary between seconds to tens of minutes or longer. Following application of the remineralizing composition in vivo, residual material can be removed by rinsing the oral cavity with water and brushing as appropriate. The remineralizing composition can be used once per day (or multiple times per day) for weeks, or months, or years until symptoms improve and dental remineralization efforts are complete. In certain embodiments, depending on the remineralizing composition's unique chemistry, the remineralization treatment may be completed in at least one dental visit (i.e. at a dental office), or at home through at least one self-application/administration.

Clinical use of the remineralizing composition in vivo has significant benefits to specific patient populations that have difficulty brushing, for example, geriatric patients, pediatric patients and patients with special needs. Additionally, the use of the remineralizing composition has significant benefits to patients that lack adequate saliva generation, for example, patients with xerostomia or undergoing certain types of chemotherapy and radiation therapy. The use of the remineralizing composition also provides sensitivity relief to patients which may be particularly useful before, during or after prophylactic dental cleanings, tooth whitening procedures, or other dental procedures. In other words, the remineralizing composition is a desensitizer that works through remineralization and occlusion of dentinal tubules (thereby minimizing sensitivity). In certain embodiments, the inclusion of potassium ions within the composition (for example, by adding potassium chloride) is advantageous as potassium ions help stop sensitivity by blocking nerve excitability. In certain embodiments, a potassium salt is included at a preferred concentration of 0.01%-1%, a more preferred concentration of 0.02%-0.5%, and a most preferred concentration of 0.04%-0.1%. Lastly, the remineralizing composition may be used as part of or following any dental procedure to aid in the remineralization of tooth structures to help maintain their integrity and strength. For example, during an endodontic procedure the remineralizing composition may be introduced into the canal prior to obturation to remineralize the tooth from within the root canal system, which may also promote sealer penetration and bonding. The clinical benefits associated with the use of the remineralizing composition result from the composition's ability to remineralize deep within the tooth structure. The depth of remineralization is preferably at least 100 nanometers, more preferably at least 1 micron, even more preferably at least 10 microns, and most preferably at least 100 microns.

In certain embodiments, the disclosed remineralization composition can be useful for non-invasive treatment of incipient caries, white spot lesions, and areas of enamel and/or dentin demineralization. While the disclosed remineralization composition is useful to treat these issues, the disclosed remineralization composition would significantly benefit other remineralization products currently on the market. Recently, bioactive peptides have been introduced on the market to help remineralize teeth and treat incipient decay and white spot lesions. An example is a product called Curodont (vVardis, Zug, Switzerland). Once applied, the bioactive peptide creates a scaffold within the tooth structure that attracts calcium and phosphate ions from saliva promoting remineralization over time. In certain instances, it may be clinically advantageous to apply the disclosed remineralization composition in vivo after the bioactive peptide to accelerate and hasten remineralization through the at least one bioactive material (e.g. apatites, apatite precursors, calcium ions, phosphate ions, and combinations thereof) contained within the disclosed remineralization composition.

Manufacturing method #1: One embodiment of a method of manufacturing the remineralizing composition is as follows and utilizes one single vessel for manufacturing: first placing water in a mixing vessel, then dissolving the at least one inorganic salt that does not contain calcium or phosphate, followed by the at least one buffering agent. Next, the pH is modified to the desired target pH using at least one pH modifier (i.e. hydrochloric acid or sodium hydroxide). Then at least one inorganic salt containing phosphate is next dissolved, followed by the at least one inorganic salt containing calcium, which results in the initiation of in-situ micro and nanosized apatite and apatite precursor particle formation (i.e. bioactive material) within the remineralizing composition. In no particular order, the at least one flavorant is then added to the mixture followed by any of the optional ingredients (e.g. sweeteners, surfactants, etc) and the thickener. The pH can be modified as needed using the at least one pH modifier during any step to particularly and specifically form certain apatites and/or apatite precursors.

Manufacturing method #2: Another embodiment of a method of manufacturing the remineralizing composition is as follows and utilizes two vessels for manufacturing: first placing water in a mixing vessel, then dissolving the at least one inorganic salt that does not contain calcium or phosphate, followed by at least one buffering agent. Next, the pH is modified to the desired target pH using at least one pH modifier (i.e. hydrochloric acid or sodium hydroxide). Then at least one inorganic salt containing calcium or phosphate is next dissolved. In a second vessel, a concentrated solution of at least one inorganic salt containing calcium or phosphate (the opposite of what was added in the first vessel) is created at the solubility limit of the inorganic salt containing calcium or phosphate. Heat may be utilized during processing to increase the solubility and concentration of the inorganic salt containing calcium within the second vessel/second mixture. The mixture from the second vessel is then added to the first vessel which results in the initiation of in-situ micro and nanosized apatite and apatite precursor particle formation (i.e. bioactive material) within the remineralizing composition. In no particular order, at least one flavorant is then added to the mixture followed by any of the optional ingredients (e.g. sweeteners, surfactants, etc) and the thickener. The pH can be modified as needed using at least one pH modifier during any step to particularly and specifically form certain apatites and/or apatite precursors.

Manufacturing method #3: Yet another embodiment of a method of manufacturing the remineralizing composition is as follows and utilizes three vessels for manufacturing: first placing water in a mixing vessel, then dissolving the at least one inorganic salt that does not contain calcium or phosphate, followed by the at least one buffering agent. Next, the pH is modified to the desired target pH using the at least one pH modifier (i.e. hydrochloric acid or sodium hydroxide). The mixture is then split into two vessels each containing equal parts of the mixture. To the first vessel, at least one inorganic salt containing calcium is dissolved and the pH is modified as needed using at least one pH modifier. To the second vessel, at least one inorganic salt containing phosphate is dissolved and the pH is modified as needed using at least one pH modifier. The mixture from the two vessels is then recombined into a third vessel which results in the initiation of in-situ micro and nanosized apatite and apatite precursor particle formation (i.e. bioactive material) within the remineralizing composition. In no particular order, at least one flavorant is then added to the mixture followed by any of the optional ingredients (e.g. sweeteners, surfactants, etc) and the thickener. The pH can be modified as needed using at least one pH modifier during any step to particularly and specifically form certain apatites and/or apatite precursors.

For any manufacturing method 1-3, it is possible to dissolve the at least one inorganic salt that does not contain calcium or phosphate after the formation of the at least one bioactive material (e.g. apatites and apatite precursors). For any manufacturing method 1-3, the final pH of the remineralization composition is critical to the formation of various apatites and apatite precursors. In certain instances, pH values between 6.5 and 8 preferentially form apatites, specifically HAp and ClAp, whereas pH values less than 6.5 preferentially form apatite precursors, specifically monetite and brushite. For any manufacturing method 1-3, the concentration of the at least one pH modifier is critical to the formation of various apatites and apatite precursors. The use of 1M (3.85%) NaOH preferentially forms HAp whereas the use of 13M (50%) NaOH preferentially forms ClHAp.

The disclosed methods of manufacturing the remineralization composition are unique and novel methods to manufacture complex apatites and apatite precursors in situ within the composition batch from simple metal salts containing calcium and phosphate. In certain embodiments the remineralization composition is comprised of one calcium containing salt and one phosphate containing salt that preferably yields at least two distinct bioactive materials within the batch, but more preferably yields at least three distinct bioactive materials within the batch. In other words, manufacturing the remineralization composition via the disclosed manufacturing processes can yield multiple distinct bioactive material products from two simple inorganic salts. In this manner, the disclosed methods are advantageous from a cost standpoint as a lower number of ingredients are needed and these ingredients are widely available and inexpensive. Additionally, the disclosed methods allow for the creation of apatites and apatite precursors through manufacturing at room temperature and atmospheric pressure.

For any manufacturing method, the pH can be modified as needed using at least one pH modifier during any step to particularly and specifically form certain apatites and/or apatite precursors.

### Definitions

For purposes of interpreting this specification, the following abbreviations, terms and definitions will apply and whenever appropriate, terms used in the singular will also include the plural and vice versa. In the event that any definition set forth below conflicts with any document incorporated herein by reference, the definition set forth below shall control.

The term “room temperature” or ambient temperature as used herein refers to common ambient temperatures ranging from about 18° C. to about 27° C.

The term “treating” refers to administering a therapy in an amount, manner, or mode effective to improve a condition, symptom, or parameter associated with a disorder. In some aspects, treating refers to the treatment of a dental ailment such as an infected tooth.

The term “substantially” as used herein means to a great or significant extent, but not completely.

As used herein, “a”, “an”, “the”, “at least one”, and “one or more” are used interchangeably.

The terms “comprises” and variations thereof do not have a limiting meaning where these terms appear in the description and claims.

The term “patient” or “subject” refers to mammals and humans. Thus, in one aspect, the subject is a mammal, or a mammal in need thereof. In one aspect, the subject is a human, or human in need thereof. In one aspect, the human or human in need thereof is a medical patient. The subject can be from ˜0 years of age to 99 years of age or older.

The term “in vivo” generally means in a living subject.

The term “in situ” generally means in place, or in its place.

The term “composition” generally refers to the chemical makeup of certain embodiments of the disclosed invention and is synonymous with “formula”, “formulation”, “solution” and the like.

The term “endodontic” generally refers to inside the tooth. The term “endodontic procedure” is generally synonymous with “root canal therapy” (abbreviated as RCT), or “root canal procedure”, and refers to a treatment of an infected tooth to cleanse the root canal system and remove the infection.

When an initial root canal treatment fails, a retreatment procedure is needed where the tooth is re-accessed and cleansed again to remove the source of infection and failure. The term “retreatment” generally refers to this type of retreatment procedure.

The term “sealer” generally refers to materials that are used as part of the obturation step of an endodontic procedure to help seal the canal. This term is generally synonymous with “endodontic sealer” and includes bioceramic sealers and non-bioceramic sealers, with bioceramic sealers mainly being cement-based materials. Within this embodiment, “sealer” is generally used synonymously with “bioceramic sealers” and refers to sealer materials that are cement-based in their composition.

The term “root canal system” generally refers to the naturally occurring anatomical spaces within the root(s) of a tooth including the pulp chamber.

The term “calcified tissue” generally refers to tissues appreciably consisting of calcium salts other calcium containing materials. Within this application, “dentinal hard tissues” are a type of “calcified tissue” and consist of enamel, dentin, and cementum.

The term “dentin” generally refers to a type of calcified tissue that is a major component of teeth. Hydroxyapatite is the main inorganic constituent of dentin and is a naturally occurring mineral from of calcium apatite with the formula Ca₅(PO₄)₃(OH).

The term “remineralizing composition” generally refers to embodiments of the present disclosure that are generally used to remineralize various tissues in vivo.

The term “demineralization” generally refers to the process of removing mineral ions from hydroxyapatite of hard tissues, for example, enamel, dentin, cementum and bone.

The term “remineralization” generally refers to the process of depositing mineral ions to hydroxyapatite of hard tissues, for example, enamel, dentin, cementum and bone. Typically these mineral ions include calcium, phosphate and fluoride, although the disclosed inventions are generally fluoride-free.

The term “apatite” generally refers to a phosphate-containing mineral. Within this disclosure, apatites include but are not limited to hydroxyapatite (HAp), carbonate substituted hydroxyapatite (CHAp), chloride substituted hydroxyapatite (ClHAp), other apatites, and combinations thereof.

The term “apatite precursors” generally refer to salts of calcium and phosphate that are intermediates to apatites. Within this disclosure, apatite precursors include but are not limited to monetite, brushite, tricalcium phosphate, tetracalcium phosphate, dicalcium phosphate, monocalcium phosphate, other calcium phosphate salts, and combinations thereof.

The term “bioactive material” generally refers to a material that elicits a biological response in vivo. Within this disclosure, example bioactive materials include, but are not limited to, bioglass, tricalcium phosphate, apatites (including hydroxyapatite (HAp), carbonate substituted hydroxyapatite (CHAp), chloride substituted hydroxyapatite (ClHAp), and other apatites), monetite, brushite, tetracalcium phosphate, dicalcium phosphate, monocalcium phosphate, other calcium phosphate salts, among others.

The above summary of the present invention is not intended to describe each disclosed embodiment or every implementation of the present invention. The description that follows more particularly exemplifies illustrative embodiments. In several places throughout the description, guidance is provided through lists of examples, which examples can be used in various combinations. In each instance, the recited list serves only as a representative group and should not be interpreted as an exclusive list.

### EXAMPLES

Table I summarizes the chemical constituents that comprise the disclosed remineralizing composition. Additionally, Table 2 provides example formulas or compositions of the disclosure including the developed and tested formulas.

TABLE 1
Chemical constituents that comprise the remineralizing composition solution.

| | | Exemplary | Exemplary | Exemplary | Exemplary |
|---|---|---|---|---|---|
| | | Concentration | Concentration | Concentration | Concentration |
| Example | Function | 1 (w/w %) | 2 (w/w %) | 3 (w/w %) | 4 (w/w %) |
| Calcium chloride | Inorganic salt | 0.1-5.0% | 0.5-3.0% | 0.1-5.0% | 10.0-37.5% |
| (CaCl2) | containing | | | | |
| | calcium | | | | |
| Monopotassium | Inorganic salt | 0.1-5.0% | 0.1-2.0% | 0.1-5.0% | 10.0-30% |
| phosphate | containing | | | | |
| (KH2PO4) | phosphate | | | | |
| Potassium | Inorganic salt | 0.1-2.5% | 1-2.5% | 0.1-2.5% | 0.1-2.5% |
| chloride (KCl) | containing | | | | |
| | neither calcium | | | | |
| | nor phosphate | | | | |
| Hydroxyapatite | Bioactive | Formed | 0% | Formed | Formed |
| formed in-situ¹ | material | in situ¹ | | in situ¹ | in situ¹ |
| Bioactive glass | Bioactive | 0% | 1-10% | 0% | 0% |
| 45S5 | material | | | | |
| TRIS buffer | Buffering agent | 0.1-2.5% | 0.1-1.5% | 0.1-1.5% | 0.1-2.5% |
| Hydrochloric | pH modifier | 0.1-5% | 0.1-5% | 0.1-5% | 0.1-5% |
| acid (HCl) or | | | | | |
| sodium | | | | | |
| hydroxide | | | | | |
| (NaOH) | | | | | |
| Methyl salicylate | Flavorant | 0-2.5% | 0.5-2.5% | 0-2.5% | 0-2.5% |
| Sodium | Sweetener | 0-2.5% | 0-2.5% | 0-2.5% | 0-2.5% |
| saccharin | | | | | |
| Poloxamer 407 | Thickener | 0-25% | 10-25% | 0-5% | 0-5% |
| Polysorbate 80 | Surfactant | 0-1% | 0.1-1% | 0.1-1% | 0.1-1% |
| Blue No. 1 | Colorant | 0-0.25% | 0.01-0.25% | 0.01-0.25% | 0.01-0.25% |
| Hydrogen | Antimicrobial | 0-2.5% | 0.5-2.5% | 0.1-2.5% | 0.1-2.5% |
| peroxide | agent | | | | |
| Ascorbic acid | Preservative | 0-5% | 0.5-5% | 0.5-5% | 0.5-5% |
| Sodium benzoate | Preservative | 0-1% | 0-1% | 0-0.25% | 0-0.25% |
| Potassium | Preservative | 0-2% | 0-2% | 0-0.75% | 0-0.75% |
| sorbate | | | | | |
| Methylparaben | Preservative | 0-0.25% | 0-0.25% | 0-0.1% | 0-0.1% |
| sodium | | | | | |
| Propylparaben | Preservative | 0-0.15% | 0-0.15% | 0-0.1% | 0-0.1% |
| sodium | | | | | |
| Water | Solvent | Balance | Balance | Balance | Balance |
| TOTAL | | 100% | 100% | 100% | 100% |
| Properties | pH (10% in DI | 3.5-8.5 | 5.0-8 | 5.0-8.0 | 5.0-8.5 |
| | water) | | | | |
| | Consistency | Gel | Gel | Liquid | Gel or liquid |
| | Appearance | Slightly | Slightly | Clear liquid | Slightly cloudy/ |
| | | cloudy/ | cloudy/ | with sediment | off white (gel) or |
| | | off white | off white | that resuspends | clear liquid with |
| | | | | when agitated | sediment that |
| | | | | | resuspends when |
| | | | | | agitated (liquid) |
| | Density (g/mL) | 0.95-1.1 | 0.95-1.1 | 0.95-1.1 | 0.95-1.25 |
| | Viscosity | 500,000- | 500,000- | 1- | 1-100 (gel) or |
| | (mPa*s) | 1,000,000 | 1,000,000 | 100 | 500,000- |
| | | | | | 2,000,000 |
| | | | | | (liquid) |

¹Hydroxyapatite formed in situ within the formulation during manufacturing due to the inorganic salt containing calcium and inorganic salt containing phosphate creating hydroxyapatite

TABLE 2
Example formulas or compositions that comprise the remineralizing solution.

| | | Formula | Formula | Formula | Formula | Formula | Formula | Formula | Formula |
|---|---|---|---|---|---|---|---|---|---|
| Chemical | | A | B | C | D | E | F | G | H |
| Component | Function | (w/w %) | (w/w %) | (w/w %) | (w/w %) | (w/w %) | (w/w %) | (w/w %) | (w/w %) |
| NaCl | Inorganic salt containing | 1.00% | 1.00% | 1.00% | 1.00% | 0.00% | 1.00% | 1.00% | 0.50% |
| | neither calcium nor | | | | | | | | |
| | phosphate | | | | | | | | |
| KCl | Inorganic salt containing | 1.50% | 1.50% | 1.50% | 1.50% | 2.50% | 1.50% | 1.50% | 0.50% |
| | neither calcium nor | | | | | | | | |
| | phosphate | | | | | | | | |
| MgCl2*6H2O | Inorganic salt containing | 0.05% | 0.05% | 0.05% | 0.05% | 0.00% | 0.05% | 0.05% | 0.10% |
| | neither calcium nor | | | | | | | | |
| | phosphate | | | | | | | | |
| CaCl2*2H2O¹ | Inorganic salt containing | 1.00% | 5.00% | 1.00% | 1.00% | 5.00% | 5.00% | 7.50% | 7.50% |
| | calcium | | | | | | | | |
| KH2PO4¹ | Inorganic salt containing | 0.35% | 2.00% | 0.35% | 0.35% | 5.00% | 2.00% | 4.00% | 4.00% |
| | phosphate | | | | | | | | |
| TRIS | Buffering agent | 0.30% | 0.50% | 0.30% | 0.30% | 0.50% | 0.30% | 0.50% | 0.50% |
| 2.5M HCl or 13M | pH modifier | 0.10% | 0.20% | 0.10% | 0.10% | 0.50% | 0.10% | 0.20% | 0.00% |
| NaOH | | | | | | | | | |
| Poloxamer 407 | Thickener | 18.00% | 18.00% | 0.00% | 20.00% | 0.00% | 20.00% | 0.00% | 0.00% |
| Polyvinyl alcohol | Thickener | 0.00% | 0.00% | 0.00% | 0.00% | 0.00% | 0.00% | 7.00% | 0.00% |
| Polyacrylic acid | Thickener & chelator | 0.00% | 0.00% | 0.00% | 0.00% | 0.00% | 0.00% | 0.00% | 5.00% |
| Methyl salicylate | Flavorant | 0.70% | 0.70% | 0.70% | 0.70% | 0.70% | 0.70% | 0.70% | 1.00% |
| Sorbitol | Sweetener | 0.00% | 10.00% | 0.00% | 10.00% | 10.00% | 7.50% | 5.00% | 2.50% |
| Xylitol | Sweetener & preservative | 0.00% | 0.00% | 0.00% | 0.00% | 0.00% | 0.50% | 0.50% | 0.10% |
| Sodium saccharin | Sweetener | 1.00% | 0.10% | 1.00% | 0.05% | 0.00% | 0.05% | 0.10% | 0.50% |
| Potassium sorbate | Preservative | 0.25% | 0.25% | 0.25% | 0.25% | 0.00% | 0.25% | 0.25% | 0.25% |
| Sodium benzoate | Preservative | 0.10% | 0.15% | 0.05% | 0.10% | 0.00% | 0.10% | 0.15% | 0.10% |
| Methylparaben | Preservative | 0.10% | 0.15% | 0.05% | 0.00% | 0.05% | 0.00% | 0.15% | 0.00% |
| sodium | | | | | | | | | |
| Propylparaben | Preservative | 0.05% | 0.10% | 0.02% | 0.00% | 0.02% | 0.00% | 0.10% | 0.00% |
| sodium | | | | | | | | | |
| Hydrogen peroxide | Antimicrobial agent | 0.00% | 0.00% | 0.00% | 1.75% | 0.00% | 1.75% | 0.00% | 0.00% |
| Polysorbate 20 | Surfactant | 0.00% | 0.00% | 0.00% | 0.20% | 0.00% | 0.20% | 0.00% | 0.00% |
| Ethanol | Solvent | 0.00% | 0.00% | 10.00% | 5.00% | 10.00% | 5.00% | 0.00% | 0.00% |
| Water | Solvent | 75.50% | 60.30% | 83.63% | 57.65% | 65.73% | 54.00% | 71.30% | 77.45% |

| | | | | | | | | |
|---|---|---|---|---|---|---|---|---|
| Total | 100.00% | 100.00% | 100.00% | 100.00% | 100.00% | 100.00% | 100.00% | 100.00% |
| Target pH | 4-8 | 4-8 | 4-8 | 4-8 | 4-8 | 4-8 | 4-8 | 4-8 |

¹At least one bioactive material (e.g. apatite, apatite precursor, and combinations thereof) is formed in situ within the formulation during manufacturing due to the inorganic salt containing calcium and inorganic salt containing phosphate creating the bioactive material; therefore, the composition contains at least one bioactive material.

### Testing

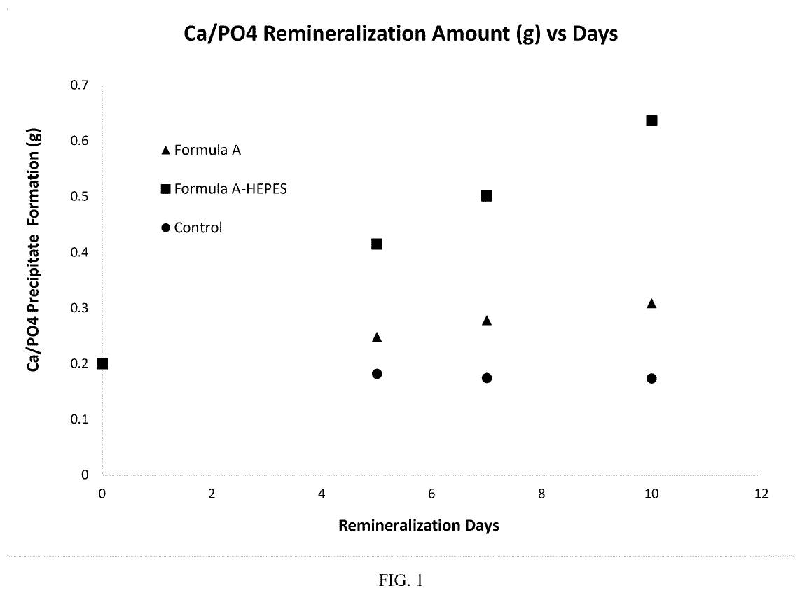

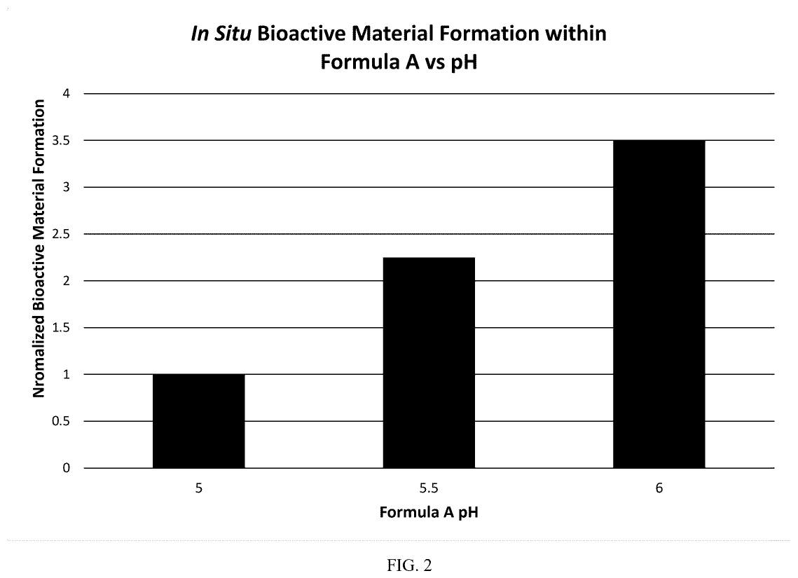

Test #1—Preliminary remineralization study: An initial test was conducted to explore the ability of the remineralizing composition to deposit calcium phosphate salts on to a hydroxyapatite powder substrate to simulate dentin. 0.2 g of hydroxyapatite powder (Sigma Aldrich, Milwaukee, WI) was added to 15 mL conical tubes along with 10 mL of Formula A. The tubes were continuously rotated for 10 days on a tube inverter. Each day the tubes were centrifuged at 330 rpm for 15 minutes to pelletize the hydroxyapatite powder and replace the supernatant/solution with fresh Formula A. At various timepoints, the mass of the pelletized hydroxyapatite powder was measured to determine mass of remineralize material deposited by Formula A. A second group of samples, which consisted of Formula A using HEPES buffer instead of TRIS buffer, was also tested along with a third group of control samples consisting of 0.2 g of hydroxyapatite powder in distilled water. Results are shown in FIG. 1 and show the significant increase in mass of the hydroxyapatite powder over the course of the experiment as a result of the remineralization composition depositing calcium phosphate salts and mineral apatites on the surface of the hydroxyapatite powder substrate. Specifically, Formula A-HEPES buffer resulted in greater than twice the amount of calcium phosphate material compared to the standard Formula A utilizing TRIS as the buffering agent. The control group (hydroxyapatite powder in distilled water) slightly decreased in mass over the course of the experiment which is likely due to accidental loss of mass from the pelletized hydroxyapatite powder when disposing of the distilled water supernatant daily. In certain embodiments, the at least one buffering agent within the remineralizing solution consists of HEPES or TRIS.
Test #2—Preliminary bioactive material formation in situ. A subsequent experiment was undertaken to assess the composition's remineralization abilities through qualitative evaluation of bioactive material formed in situ within the remineralization composition, while also investigating the impact of Formula A's pH (5, 5.5, 6). Results are shown in FIG. 2 and illustrate that more bioactive material is formed in situ as the pH of Formula A is increased. Therefore, in certain embodiments it may be advantageous for the remineralizing composition to have a slightly acidic pH (5-6.5) or more neutral pH (˜7).
Test #3—Preliminary XRD analysis of bioactive material formed in situ. In an effort to characterize the bioactive material created in situ within the previous tests another test was performed following the same methodology as Test #1 but hydroxyapatite powder was not included as a substrate. Therefore, over the course of ten days the bioactive material formed in situ within the remineralizing composition was pelletized and kept growing in mass as fresh Formula A (pH 4.5) was replenished daily. Within this test, Formula A using HEPES as the buffering agent instead of TRIS was also used as an experimental test group. Following ten days, the bioactive materials from Formula A and Formula A-HEPES were isolated and sent to Intertek (Trexlertown, PA) for x-ray diffraction (XRD) analysis along with a hydroxyapatite powder control (Sigma Aldrich, Milwaukee, WI). Results are shown in Table 3 and illustrate that the in situ formed bioactive material is predominantly a blend of monetite and hydroxyapatite. Results also confirm that significantly more hydroxyapatite is formed in the Formula A composition using TRIS as the buffering agent compared to the Formula A composition using HEPES as the buffering agent. Surprisingly, and not identified in the prior art or literature, a significant amount of hydroxyapatite is formed even though the pH of Formula A in this test was quite acidic at a pH of 4.5. This is a unique property of the disclosed remineralizing compositions in that a significant amount of hydroxyapatite is still formed at acidic pHs. In certain embodiments, the in situ formed bioactive material is comprised of calcium phosphate salts, including apatites, which consist of preferably at least 30% hydroxyapatite, more preferably at least 50% hydroxyapatite, and most preferably at least 65% hydroxyapatite. In certain embodiments, the in situ formed bioactive material is comprised of apatite precursors consist of preferably at least 5% calcium phosphate salts, more preferably at least 30% calcium phosphate salts, and most preferably at least 50% calcium phosphate salts.

TABLE 3
XRD results of the in situ formed bioactive material
for material identification/characterization

| | | Calculated w/w % In each Sample | | |
|---|---|---|---|---|
| Compound/ | | | Formula | Hydroxyapatite |
| mineral name | Formula | Formula A | A-HEPES | (HA) powder |
| Monetite | CaHPO₄ | 31.6% | 50.5% | 8.4% |
| Hydroxyapatite | Ca₅(PO₄)₃(OH) | 67.7% | 47.8% | 91.6% |
| Halite | NaCl | 0.6% | 1.1% | — |
| Hydrogen | Na₂HPO₄ | — | 0.5% | — |
| Sodium | | | | |
| Phosphate | | | | |

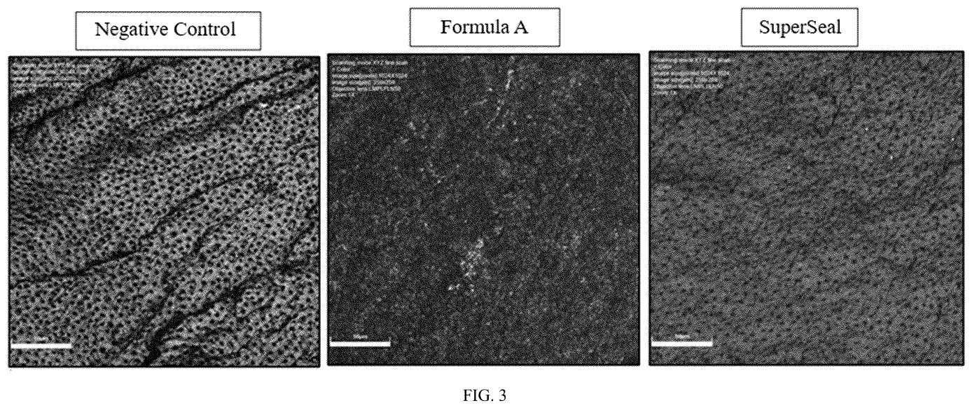

Test #4—Ex vivo dentinal tubule occlusion study. An ex vivo dentinal tubule occlusion study was performed on extracted human teeth to evaluate the remineralizing composition's ability to occlude open dentinal tubules (which is caused by demineralization and results in tooth sensitivity). Methods from the literature were used on the extracted teeth to demineralize the teeth using citric acid (J Clin Periodontol. 1993 May; 20 (5): 366-70). Demineralized teeth were placed in a centrifuge tube and exposed to different materials (5 mL of Formula A or 1 mL of SuperSeal in accordance with the product's instructions for use (Pheonix Dental, Fenton, MI)) for 30 minutes on a tube inverter. Teeth were removed from the materials, rinsed with distilled water, and then stored in distilled water. This process was completed once per day for seven days. A negative control was implemented in this study which consisted of demineralized teeth that were kept in distilled water for the duration of the experiment. Teeth were then imaged using a laser microscope (LEXT OLS4000, Olympus). Images from the study were graded by blinded personnel following the proposed rating system by Hulsmann et al (J Endod. 1997 May; 23 (5): 301-6.) where a score of “0” indicates dentinal tubules are completely open, a score of “1” indicates that ˜25% of the dentinal tubules are closed, a score of “2” indicates that ˜50% of the dentinal tubules are closed, a score of “3” indicates that ˜75% of the dentinal tubules are closed, and a score of “4” indicates that 100% of the dentinal tubules are closed. Results are shown in Table 4 and representative images are shown in FIG. 3. The results conclude that Formula A results in significantly greater dentinal occlusion than SuperSeal.

TABLE 4
Average grading score results from dentinal tubule occlusion
study using various remineralizing solutions

| Sample | Average Score | SD | CV |
|---|---|---|---|
| Negative Control | 0.0 | 0.0 | 0.0 |
| Formula A | 3.4 | 1.0 | 0.3 |
| SuperSeal | 1.4 | 0.9 | 0.6 |

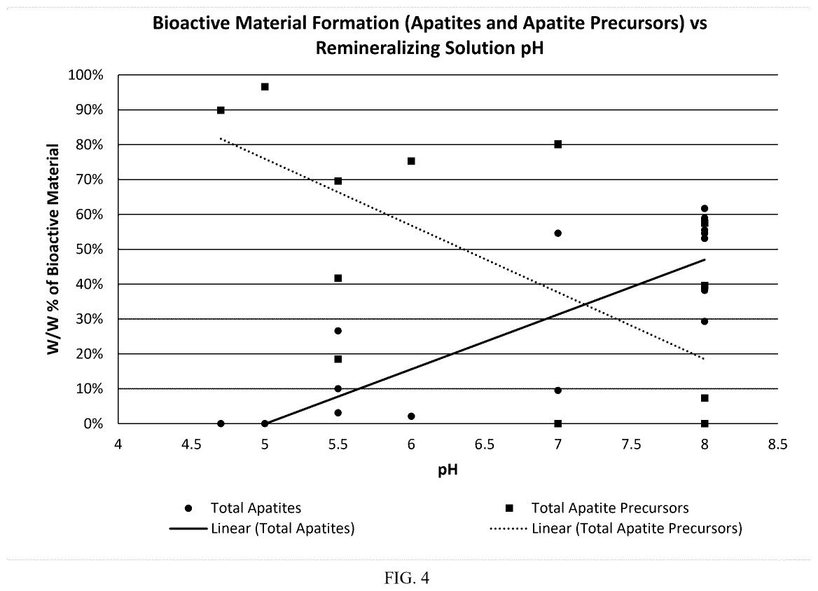

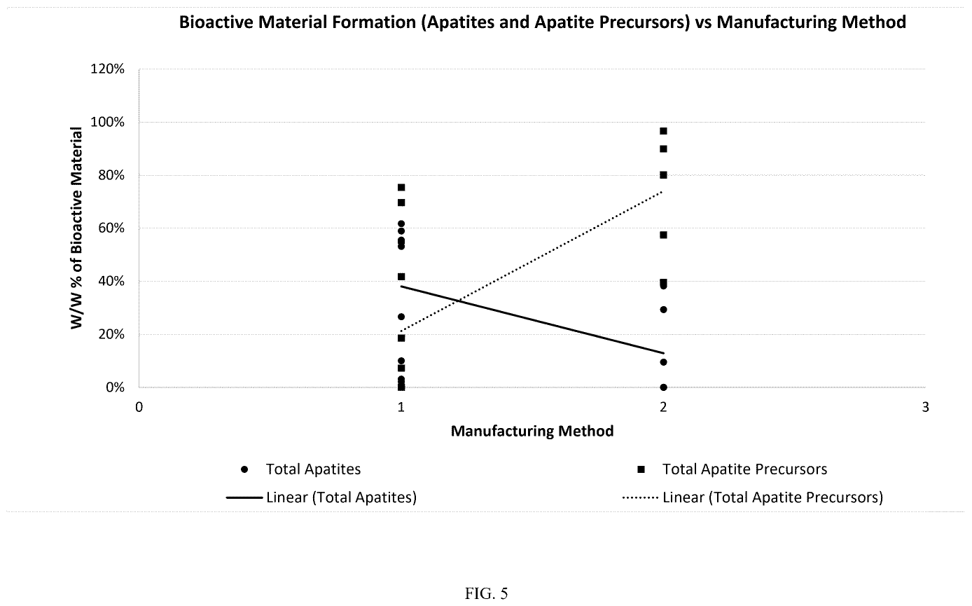

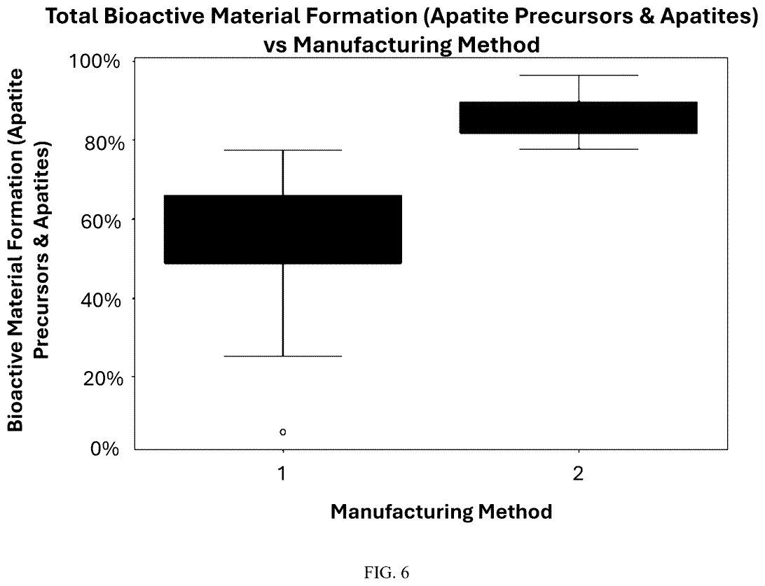

Test #5—Preliminary patient desensitization in vivo. To evaluate the ability of the disclosed remineralizing composition to help patients suffering from tooth sensitivity, Formula A samples were sent to two patients for use within custom PerioTrays® A bead of Formula A was applied within the PerioTrays® and the trays were worn by the patients for 15 minutes daily for one week. Throughout each day the patients recorded their tooth sensitivity levels using a visual analogue scale (VAS). After two applications, both patients reported total cessation of sensitivity to hot, cold, sweet, and acidic foods and beverages. Patients continued with the Formula A application for the remainder of the week and overall reported positive feedback. Subsequent follow-up by the patients reported that their sensitivity remained absent for 4-6 weeks after the one week of using Formula A for 15 minutes per day. As such, in certain embodiments the remineralizing solution provides sensitivity relief within 30 minutes of use and benefits to the patient even after treatment stops.
Test #6—Expanded XRD testing of bioactive material formed in situ. To further investigate the formation of apatite precursors and apatites in situ within the remineralizing composition, solid precipitate samples from the formulations listed in Table 5 were sent to Intertek (Trexlertown, PA) for x-ray diffraction (XRD) analysis to identify the composition of the formed biomaterials (i.e. apatite precursors and apatites). For ease of testing, the tested remineralizing compositions did not include any thickening agents as these are not expected to have any impact on the formation of apatite precursors or apatites. Table 6 shows XRD results for the various formulas. In general, more apatites were formed at pHs between 7-8 whereas more acidic pHs led to more apatite precursor formation (FIG. 4). Surprisingly, the simple inclusion of preservatives potassium sorbate and sodium benzoate (Formula 2) led to significantly more apatite formation to an equivalent remineralizing composition without these preservatives (Formula 1). This discovery is unique and novel to this disclosure. However, other preservatives, for example parabens, did not seem to appreciably impact apatite or apatite precursor formation. Interestingly, the method of manufacturing had a significant impact on apatite precursor and apatite formation, with manufacturing method #2 resulting in the formation of more apatite precursor formation whereas manufacturing method #1 resulted in the formation of more apatite formation (FIG. 5). The method of manufacturing had a significant impact on total bioactive material formation as well, with manufacturing method #2 resulting in the formation of more total bioactive material formation compared to manufacturing method #1 (FIG. 6). Surprisingly, the method pH adjusting also impacted the formation of specific apatites. Formula 14 and Formula 13 are identical except Formula 14 utilized 1M NaOH for pH adjusting while Formula 13 used 13M NaOH for pH adjusting. When the lower concentration of 1M NaOH was used (Formula 14), more hydroxyapatite was formed while the higher concentration of 13M NaOH (Formula 13) resulted in the formation of more chlorine-substituted hydroxyapatite. Both formulas resulted in nearly identical total apatite formation (53-55%) overall. Lastly, the data supports stability of the remineralizing composition and a 30-month shelf life given that the Formula 1 30 month accelerated aged sample still resulted in 29% bioactive materials including apatite precursors and apatites.

Depending on the clinical use of the remineralizing solution, in preferred embodiments the remineralizing solution is preferably at a pH between 7-8 to maximize the amount of in situ apatite formation (HAp and ClHAp); however, a pH between 5.5-7 may be preferred in certain applications where apatite precursors are deemed advantageous. The method of manufacturing the remineralizing solution composition can also be specifically tailored to preferentially form apatites, apatite precursors, or combinations thereof. Specifically, the disclosed manufacturing method #1 is preferred for more apatite formation whereas manufacturing method #2 is preferred for more apatite precursor formation. Overall, manufacturing method #2 resulted in higher bioactive material (i.e. apatites plus apatite precursors) formation in situ than manufacturing method #1. Additionally, the use of different concentrations of NaOH for pH adjusting can be easily employed to preferentially form apatites, apatite precursors, or combinations thereof. Specifically, lower concentrations of NaOH may be preferred to preferentially form HAp while higher concentrations of NaOH may be preferred to form ClHAp. In certain instances, the remineralizing solution comprises potassium sorbate, sodium benzoate, or combinations thereof which aid in the formation of apatites, and specifically HAp.

| | | Formula | Formula | Formula | Formula | Formula | Formula | Formula | Formula |
|---|---|---|---|---|---|---|---|---|---|
| Chemical | | 1 | 2 | 3 | 4 | 5 | 6 | 7 | 8 |
| Component | Function | (w/w %) | (w/w %) | (w/w %) | (w/w %) | (w/w %) | (w/w %) | (w/w %) | (w/w %) |
| NaCl | Inorganic salt | 1.00% | 1.00% | 1.00% | 1.00% | 1.00% | 1.00% | 1.00% | 1.00% |
| | containing neither | | | | | | | | |
| | calcium nor phosphate | | | | | | | | |
| KCl | Inorganic salt | 1.50% | 1.50% | 1.50% | 1.50% | 1.50% | 1.50% | 1.50% | 1.50% |
| | containing neither | | | | | | | | |
| | calcium nor phosphate | | | | | | | | |
| MgCl₂*6H₂O | Inorganic salt | 0.05% | 0.05% | 0.05% | 0.05% | 0.05% | 0.05% | 0.05% | 0.05% |
| | containing neither | | | | | | | | |
| | calcium nor phosphate | | | | | | | | |
| CaCl₂*2H₂O | Inorganic salt | 1.00% | 1.00% | 1.00% | 1.00% | 1.00% | 1.00% | 1.00% | 1.00% |
| | containing calcium | | | | | | | | |
| KH₂PO₄ | Inorganic salt | 0.35% | 0.35% | 0.35% | 0.35% | 0.35% | 0.35% | 0.35% | 0.35% |
| | containing phosphate | | | | | | | | |
| TRIS | Buffering agent | 0.30% | 0.30% | 0.30% | 0.30% | 0.30% | 0.30% | 0.30% | 0.30% |
| Potassium | Preservative | 0.00% | 0.25% | 0.00% | 0.00% | 0.00% | 0.00% | 0.00% | 0.00% |
| sorbate | | | | | | | | | |
| Sodium | Preservative | 0.00% | 0.10% | 0.00% | 0.00% | 0.00% | 0.00% | 0.00% | 0.00% |
| benzoate | | | | | | | | | |
| Methylparaben | Preservative | 0.00% | 0.00% | 0.00% | 0.00% | 0.00% | 0.00% | 0.00% | 0.00% |
| sodium | | | | | | | | | |
| Propylparaben | Preservative | 0.00% | 0.00% | 0.00% | 0.00% | 0.00% | 0.00% | 0.00% | 0.00% |
| sodium | | | | | | | | | |
| Water | Solvent | 95.80% | 95.45% | 95.80% | 95.80% | 95.80% | 95.80% | 95.80% | 95.80% |

| | | | | | | | | |
|---|---|---|---|---|---|---|---|---|
| Total | 100.00% | 100.00% | 100.00% | 100.00% | 100.00% | 100.00% | 100.00% | 100.00% |
| Target pH | 5.5 | 5.5 | 6 | 7 | 8 | 4.7 | 7 | 8 |
| (use HCl or NaOH) | | | | | | | | |
| pH after or during | After | After | After | After | After | After | After | After |
| mix of Ca and PO₄ | | | | | | | | |
| containing vessels | | | | | | | | |
| Manufacturing | Method 1 | Method 1 | Method 1 | Method 1 | Method 1 | Method 2 | Method 2 | Method 2 |
| process | | | | | | | | |

| | | Formula | Formula | Formula | Formula | Formula | Formula | Formula |
|---|---|---|---|---|---|---|---|---|
| Chemical | | 9 | 10 | 11 | 12 | 13 | 14 | 15 |
| Component | Function | (w/w %) | (w/w %) | (w/w %) | (w/w %) | (w/w %) | (w/w %) | (w/w %) |
| NaCl | Inorganic salt | 1.00% | 1.00% | 1.00% | 1.00% | 1.00% | 1.00% | 1.00% |
| | containing neither | | | | | | | |
| | calcium nor phosphate | | | | | | | |
| KCl | Inorganic salt | 1.50% | 1.50% | 1.50% | 1.50% | 1.50% | 1.50% | 1.50% |
| | containing neither | | | | | | | |
| | calcium nor phosphate | | | | | | | |
| MgCl₂*6H₂O | Inorganic salt | 0.05% | 0.05% | 0.05% | 0.05% | 0.05% | 0.05% | 0.05% |
| | containing neither | | | | | | | |
| | calcium nor phosphate | | | | | | | |
| CaCl₂*2H₂O | Inorganic salt | 1.00% | 1.00% | 1.00% | 1.00% | 1.00% | 1.00% | 1.00% |
| | containing calcium | | | | | | | |
| KH₂PO₄ | Inorganic salt | 0.35% | 0.35% | 0.35% | 0.35% | 0.35% | 0.35% | 0.35% |
| | containing phosphate | | | | | | | |
| TRIS | Buffering agent | 0.30% | 0.30% | 0.30% | 0.30% | 0.30% | 0.30% | 0.30% |
| Potassium | Preservative | 0.25% | 0.25% | 0.00% | 0.75% | 0.00% | 0.00% | 0.25% |
| sorbate | | | | | | | | |
| Sodium | Preservative | 0.10% | 0.10% | 0.00% | 0.00% | 0.00% | 0.00% | 0.10% |
| benzoate | | | | | | | | |
| Methylparaben | Preservative | 0.00% | 0.00% | 0.00% | 0.00% | 0.06% | 0.06% | 0.06% |
| sodium | | | | | | | | |
| Propylparaben | Preservative | 0.00% | 0.00% | 0.00% | 0.00% | 0.04% | 0.04% | 0.04% |
| sodium | | | | | | | | |
| Water | Solvent | 95.45% | 95.45% | 95.80% | 95.05% | 95.70% | 95.70% | 95.35% |

| | | | | | | | |
|---|---|---|---|---|---|---|---|
| Total | 100.00% | 100.00% | 100.00% | 100.00% | 100.00% | 100.00% | 100.00% |
| Target pH | 7 | 8 | 5 | 8 | 8 | 8* | 8 |
| (use HCl or NaOH) | | | | | | | |
| pH after or during | After | After | After | After | After | After | During |
| mix of Ca and PO₄ | | | | | | | |
| containing vessels | | | | | | | |
| Manufacturing | Method 2 | Method 2 | Method 2 | Method 1 | Method 1 | Method 1 | Method 1 |
| process | | | | | | | |

*Used 1M NaOH instead of 13M NaOH for pH adjustment
5: Remineralizing composition formulas sent for x-ray diffraction (XRD) analysis w 27 Test #6.

TABLE 6
Weight/Weight % Concentration of Bioactive Materials within Solids
Obtained from Remineralizing Composition Formulas in Table 5.

| | | | | | | | Total |
|---|---|---|---|---|---|---|---|
| | | | Apatites | | | | Bioactive |
| | | | | Chlorine- | | | Materials |
| | | | | substituted | Total | | (Apatite |
| | Apatite Precursors | | Hydroxyapatite | hydroxyapatite | Apatite | Total | Precursors + |
| Formula | Brushite | Monetite | (HAp) | (ClHAp) | Precursors | Apatites | Apatites) |
| Formula 1 | 0% | 70% | 0% | 3% | 70% | 3% | 73% |
| Formula 1 | 0% | 19% | 10% | 0% | 19% | 10% | 29% |
| (accelerated | | | | | | | |
| aged to 30 | | | | | | | |
| months) | | | | | | | |
| Formula 2 | 0% | 42% | 27% | 0% | 42% | 27% | 68% |
| Formula 3 | 29% | 47% | 2% | 0% | 75% | 2% | 77% |
| Formula 4 | 0% | 0% | 55% | 0% | 0% | 55% | 55% |
| Formula 5 | 0% | 0% | 56% | 0% | 0% | 56% | 56% |
| Formula 6 | 0% | 90% | 0% | 0% | 90% | 0% | 90% |
| Formula 7 | 53% | 27% | 0% | 0% | 80% | 0% | 80% |
| Formula 8 | 19% | 21% | 0% | 38% | 40% | 38% | 78% |
| Formula 9 | 37% | 44% | 0% | 10% | 80% | 10% | 90% |
| Formula 10 | 3% | 55% | 0% | 29% | 58% | 29% | 87% |
| Formula 11 | 0% | 97% | 0% | 0% | 97% | 0% | 97% |
| Formula 12 | 7% | 0% | 34% | 25% | 7% | 59% | 66% |
| Formula 13 | 0% | 0% | 5% | 50% | 0% | 55% | 55% |
| Formula 14 | 0% | 0% | 39% | 14% | 0% | 53% | 53% |
| Formula 15 | 0% | 0% | 27% | 35% | 0% | 62% | 62% |

Test #7—Analysis of percent solids of the bioactive material within the remineralization solution. Remineralization solution (Formula 15) was made following manufacturing method #1 and the solution was pH adjusted to 8 after mixing of the calcium and phosphate containing vessels. Solids from the remineralization solution were obtained by filtering the solution through a Buchner funnel with a 20 μm filter attached to a vacuum pump. The solids on the filter were dried in a desiccator for 14 days and the mass was compared to the total mass of the remineralization solution, which yielded a percent solids value of 1.1%. In certain embodiments, the remineralization solution is comprised of preferably between 0.5%-35% bioactive material solids (i.e. apatite and apatite precursors), more preferably between 1%-10% bioactive material solids, even more preferably between 1%-5% bioactive material solids, and most preferably between 1%-2% bioactive material solids. Considering that the calcium and phosphate salts account for 1.35% of the total mass of Formula 15, the conversion of the raw calcium and phosphate salts to bioactive materials is fairly high at 81%. In certain embodiments, the conversion of the at least one inorganic salt containing calcium and the at least one inorganic salt containing phosphate into bioactive material (i.e. apatites and apatite precursors) in situ within the remineralizing solution batch is preferably at least 10%, more preferably at least 30%, even more preferably at least 50%, and most preferably at least 75%.

Within the remineralizing solution, the in situ bioactive material formation comprises at least one apatite precursor, at least one apatite, and combinations thereof. In certain embodiments, the at least one apatite precursor comprises brushite, monetite, or combinations thereof and is at a weight/weight percent concentration of preferably between 0.01%-25.0%, more preferably between 0.05%-10.0%, even more preferably between 0.1%-5.0%, and most preferably between 0.1%-2.5% within the remineralizing solution. In certain embodiments, at least one apatite comprises hydroxyapatite (HAp), chlorine-substituted hydroxyapatite (ClHAp), or combinations thereof and is at a weight/weight percent concentration of preferably between 0.01%-25.0%, more preferably between 0.05%-10.0%, even more preferably between 0.1%-5.0%, and most preferably between 0.1%-2.5% within the remineralizing solution.
Test #8—ICP-OES testing. To evaluate the amount of free calcium and phosphate present in the remineralization compositions following the in situ formation of the bioactive materials, inductively coupled plasma optical emission spectrometry (ICP-OES) testing was performed on supernatant samples from remineralization compositions listed in Table 7. Briefly, compositions were centrifuged at 300 RPM to separate the formed bioactive materials from the supernatant. 15 mL of supernatant was obtained for each sample and sent to ATI-Aquaristik GmbH (Hamm, Germany). Results are shown in Table 8 and demonstrate the presence of free ions. Formulas 16 and 18 are only calcium containing controls, while Formulas 17 and 19 are only phosphate containing controls. In certain embodiments, free calcium ions are present within the remineralizing composition preferably between 100 parts per billion (ppb) and 10000 ppb, more preferably between 300 ppb and 5000 ppb, and most preferably between 500 ppb and 1000 ppb. In certain embodiments, free phosphate ions are present within the remineralizing composition preferably between 10 parts per trillion (ppt) and 5000 ppb, more preferably between 100 ppt and 1000 ppb, even more preferably between 500 ppt and 100 ppb, and most preferably between 1 ppb and 500 ppb. In certain embodiments, free potassium ions are present within the remineralizing composition preferably between 10 ppb and 100 parts per million (ppm), more preferably between 100 ppb and 10 ppm, and most preferably between 1 ppm and 10 ppm.

| | | Formula | Formula | Formula | Formula | Formula | Formula |
|---|---|---|---|---|---|---|---|
| Chemical | | 16 | 17 | 18 | 19 | 20 | 21 |
| Component | Function | (w/w %) | (w/w %) | (w/w %) | (w/w %) | (w/w %) | (w/w %) |
| NaCl | Inorganic salt | 1.00% | 1.00% | 1.00% | 1.00% | 1.00% | 1.00% |
| | containing neither | | | | | | |
| | calcium nor phosphate | | | | | | |
| KCl | Inorganic salt | 1.50% | 1.50% | 1.50% | 1.50% | 1.50% | 1.50% |
| | containing neither | | | | | | |
| | calcium nor phosphate | | | | | | |
| MgCl₂*6H₂O | Inorganic salt | 0.05% | 0.05% | 0.05% | 0.05% | 0.05% | 0.05% |
| | containing neither | | | | | | |
| | calcium nor phosphate | | | | | | |
| CaCl₂*2H₂O | Inorganic salt | 2.00% | 0.00% | 2.00% | 0.00% | 1.00% | 1.00% |
| | containing calcium | | | | | | |
| KH₂PO₄ | Inorganic salt | 0.00% | 0.70% | 0.00% | 0.70% | 0.35% | 0.35% |
| | containing phosphate | | | | | | |
| TRIS | Buffering agent | 0.30% | 0.30% | 0.30% | 0.30% | 0.30% | 0.30% |
| Potassium | Preservative | 0.25% | 0.25% | 0.00% | 0.00% | 0.25% | 0.00% |
| sorbate | | | | | | | |
| Sodium | Preservative | 0.10% | 0.10% | 0.00% | 0.00% | 0.10% | 0.00% |
| benzoate | | | | | | | |
| Methylparaben | Preservative | 0.06% | 0.06% | 0.00% | 0.00% | 0.06% | 0.00% |
| sodium | | | | | | | |
| Propylparaben | Preservative | 0.04% | 0.04% | 0.00% | 0.00% | 0.04% | 0.00% |
| sodium | | | | | | | |
| Water | Solvent | 94.70% | 96.00% | 95.15% | 96.45% | 95.35% | 95.80% |
| Total | | 100.00% | 100.00% | 100.00% | 100.00% | 100.00% | 100.00% |
| Target pH | | 7.4 | 7.4 | 7.4 | 7.4 | 6 | 6 |
| (use HCl or NaOH) | | | | | | | |
| pH after or during | | N/A | N/A | N/A | N/A | After | After |
| mix of Ca and PO₄ | | | | | | | |
| containing vessels | | | | | | | |
| Manufacturing | | N/A | N/A | N/A | N/A | Method 1 | Method 1 |
| process | | | | | | | |
| Addition of NaOH | | N/A | N/A | N/A | N/A | Fast | Fast |
| (slow vs fast) | | | | | | | |
| Notes | | Only | Only | Only | Only | | |
| | | calcium | phosphate | calcium | phosphate | | |
| | | containing | containing | containing | containing | | |
| | | as a | as a | as a | as a | | |
| | | reference | reference | reference | reference | | |
| | | Formula | Formula | Formula | Formula | Formula | Formula |
| Chemical | | 22 | 23 | 23 | 25 | 26 | 27 |
| Component | Function | (w/w %) | (w/w %) | (w/w %) | (w/w %) | (w/w %) | (w/w %) |
| NaCl | Inorganic salt | 1.00% | 1.00% | 1.00% | 1.00% | 1.00% | 1.00% |
| | containing neither | | | | | | |
| | calcium nor phosphate | | | | | | |
| KCl | Inorganic salt | 1.50% | 1.50% | 1.50% | 1.50% | 1.50% | 1.50% |
| | containing neither | | | | | | |
| | calcium nor phosphate | | | | | | |
| MgCl₂*6H₂O | Inorganic salt | 0.05% | 0.05% | 0.05% | 0.05% | 0.05% | 0.05% |
| | containing neither | | | | | | |
| | calcium nor phosphate | | | | | | |
| CaCl₂*2H₂O | Inorganic salt | 1.00% | 1.00% | 1.00% | 1.00% | 1.00% | 1.00% |
| | containing calcium | | | | | | |
| KH₂PO₄ | Inorganic salt | 0.35% | 0.35% | 0.35% | 0.35% | 0.35% | 0.35% |
| | containing phosphate | | | | | | |
| TRIS | Buffering agent | 0.30% | 0.30% | 0.30% | 0.30% | 0.30% | 0.30% |
| Potassium | Preservative | 0.25% | 0.25% | 0.25% | 0.25% | 0.25% | 0.00% |
| sorbate | | | | | | | |
| Sodium | Preservative | 0.10% | 0.10% | 0.10% | 0.10% | 0.10% | 0.00% |
| benzoate | | | | | | | |
| Methylparaben | Preservative | 0.06% | 0.06% | 0.06% | 0.06% | 0.06% | 0.00% |
| sodium | | | | | | | |
| Propylparaben | Preservative | 0.04% | 0.04% | 0.04% | 0.04% | 0.04% | 0.00% |
| sodium | | | | | | | |
| Water | Solvent | 95.35% | 95.35% | 95.35% | 95.35% | 95.35% | 95.80% |

| | | | | | | |
|---|---|---|---|---|---|---|
| Total | 100.00% | 100.00% | 100.00% | 100.00% | 100.00% | 100.00% |
| Target pH | 8 | 6 | 8 | 6 | 8 | 8 |
| (use HCl or NaOH) | | | | | | |
| pH after or during | After | After | During | During | After | After |
| mix of Ca and PO₄ | | | | | | |
| containing vessels | | | | | | |
| Manufacturing | Method 1 | Method 1 | Method 1 | Method 1 | Method 1 | Method 1 |
| process | | | | | | |
| Addition of NaOH | Slow | Slow | Slow | Slow | Fast | Fast |
| (slow vs fast) | | | | | | |
| Notes | | | | | | |

7: Remineralizing composition formulas sent for inductively coupled plasma (ICP-OES) analysis within Test #8.

TABLE 8
Inductively coupled plasma (ICP-OES) testing results.

| | Calcium | | Phosphate | | Potassium | | Chloride | |
|---|---|---|---|---|---|---|---|---|
| Formulas | w/w % | ppb | w/w % | ppb | w/w % | ppm | w/w % | ppm |
| Formula 16 | 0.034% | 3361 | 0.000% | 0.7 | 0.059% | 5.908 | 1.774% | 177.35 |
| Formula 17 | 0.001% | 146.5 | 0.032% | 3163 | 0.067% | 6.719 | 1.552% | 155.15 |
| Formula 18 | 0.031% | 3109 | 0.000% | 0.2 | 0.052% | 5.154 | 1.734% | 173.44 |
| Formula 19 | 0.001% | 147 | 0.031% | 3129 | 0.070% | 7.043 | 1.589% | 158.86 |
| Formula 20 | 0.009% | 927.1 | 0.000% | 33.8 | 0.070% | 7.016 | 1.637% | 163.66 |
| Formula 21 | 0.008% | 844.9 | 0.000% | 49 | 0.062% | 6.182 | 1.625% | 162.46 |
| Formula 22 | 0.008% | 771.4 | 0.000% | 13.7 | 0.065% | 6.46 | 1.639% | 163.94 |
| Formula 23 | 0.009% | 923.7 | 0.000% | 28.2 | 0.070% | 7.009 | 1.635% | 163.53 |
| Formula 24 | 0.006% | 635.4 | 0.000% | 5.8 | 0.059% | 5.866 | 1.594% | 159.39 |
| Formula 25 | 0.009% | 929.3 | 0.000% | 35.4 | 0.069% | 6.942 | 1.620% | 162 |
| Formula 26 | 0.007% | 664.5 | 0.000% | 9.6 | 0.064% | 6.443 | 1.644% | 164.39 |
| Formula 27 | 0.009% | 883 | 0.000% | 2.3 | 0.062% | 6.155 | 1.687% | 168.68 |

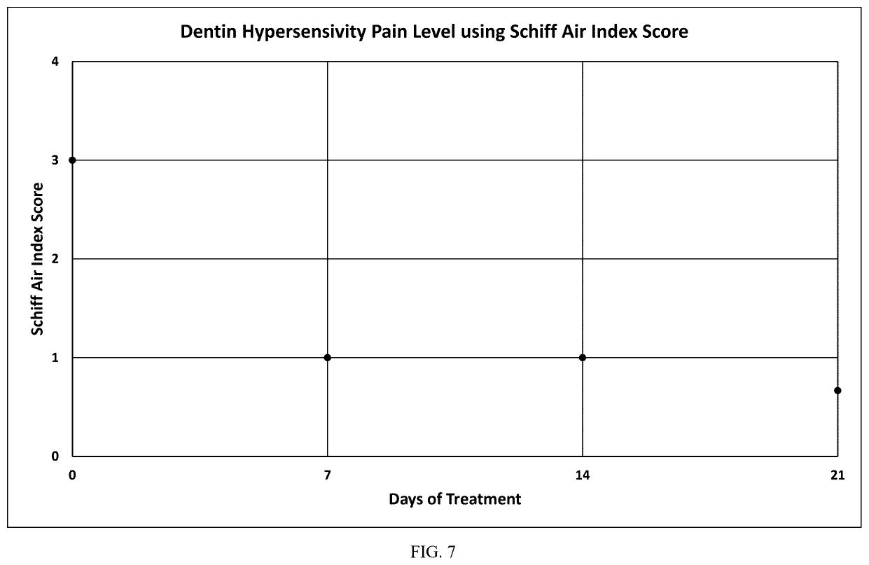

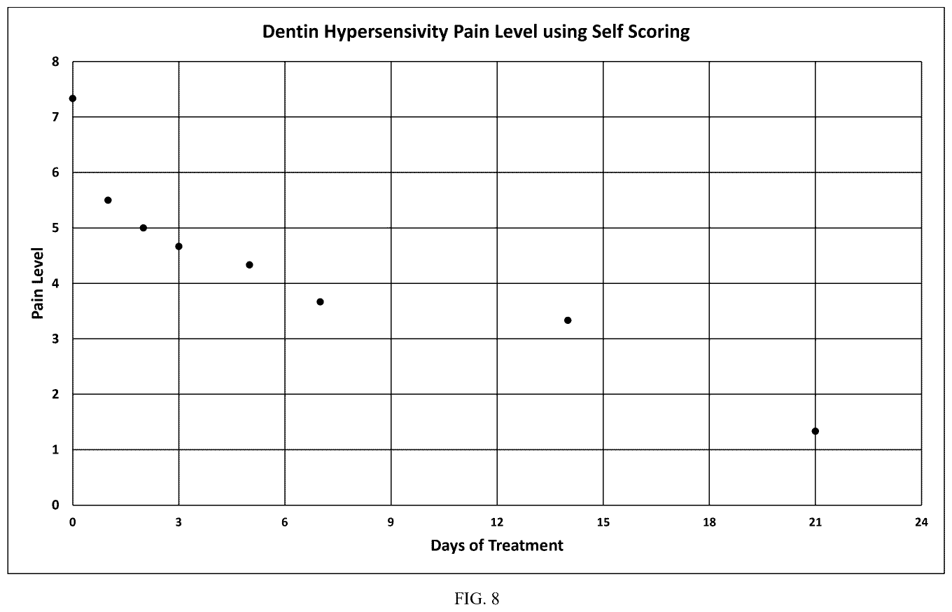

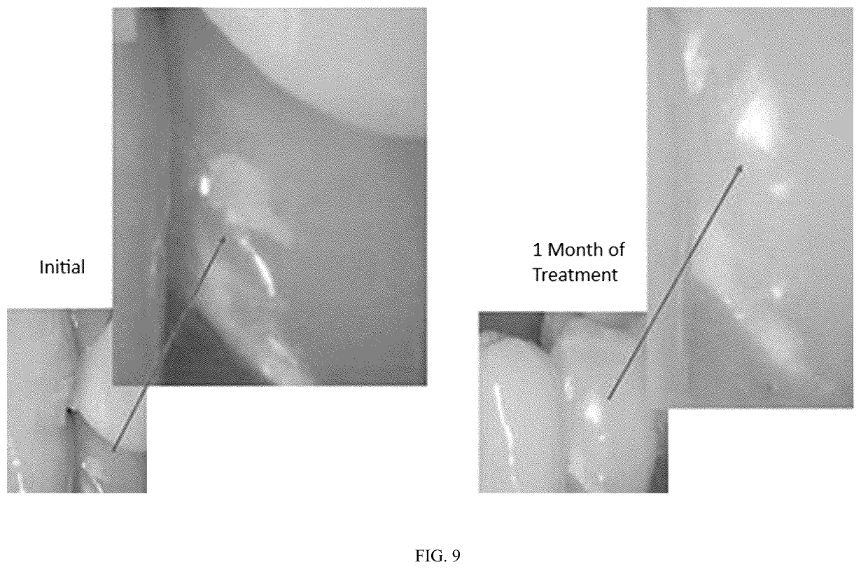

Test #9—Additional patient desensitization in vivo: Dentin hypersensitivity cases caused by generalized gum recession due to periodontitis were treatment using the remineralizing solution. Patients were administered the remineralizing solution composition (Formula 15) daily for 15 minutes using a PerioTray® for 21 days. Evaluation of tooth sensitivity was performed using the Air Schiff Index score by the clinician and Visual Analog Scale (VAS) by the patient. Air Schiff Index score scale is based on a 0-3 scale (Score 0: Tooth/Subject does not respond to air stimulus, Score 1: Tooth/Subject responded to the air stimulus but did not request discontinuation of the stimulus, Score 2: Tooth/Subject responded to the air stimulus and requested discontinuation or moved from the stimulus, Score 3: Tooth/Subject responded to the air stimulus, considered the stimulus to by painful, and requested discontinuation of the stimulus). Self-scoring pain scale using VAS is based on 0-10 (0 being no pain and 10 being worst possible pain). FIG. 7. And FIG. 8. shows the average pain level experienced dramatically decreased over the duration of the study when using the remineralizing solution. One patient went from a VAS self-score pain of 5 before treatment, which went down to a score of 1 after one treatment, and a score of zero (0) after the second treatment. This patient had an Air Schiff Index score of 3 before treatment which went down to a score of 0 after one week. In certain embodiments, the remineralizing solution decreases patient dentin hypersensitivity preferably within three weeks of treatment, more preferably within one week of treatment, and most preferably within one day of treatment. In certain embodiments, the remineralizing solution decreases patient dentin hypersensitivity preferably within one 15 minute application.
Test #10—White spot lesion (i.e. demineralization) treatment using the remineralizing solution: FIG. 9 shows a representative case study using the disclosed remineralizing solution to treat an area of demineralization (e.g. white spot lesion) on a patient's enamel. Here the patient applied the remineralizing solution composition (Formula 15) daily for 15 minutes using a PerioTray® for one month. As is shown in the figure, the white spot lesion is completely gone as the remineralizing solution has successfully remineralized the demineralized enamel.

### Statements

The following statements are illustrative and within the scope of the embodiments of the invention described herein.
1. A dental remineralizing composition, comprising:
   at least two inorganic salts,
   at least one bioactive material,
   at least one buffering agent,
   at least one solvent, and,
   wherein at least a first inorganic salt yields free calcium (Ca²⁺) ions in water, and
   wherein at least a second inorganic salt yields free phosphate ions in water.
2. The composition of statement 1, further comprising one or more of a thickener, a flavorant, a sweetener, a surfactant, a hydrotrope, a colorant, an antimicrobial agent, a preservative, a prebiotic, a probiotic, an antioxidant, a chelator, or a sequestrant,
3. The composition of statement 1, wherein the bioactive material is formed in situ during manufacturing as a result of a chemical reaction between the first inorganic salt containing calcium and the second inorganic salt containing phosphate.
4. The composition of statement 1, wherein the conversion of the at least one inorganic salt containing calcium and the at least one inorganic salt containing phosphate into at least one bioactive material in situ within the remineralizing solution batch is at least 30%.
5. The composition of statement 1, wherein the bioactive material is formed in situ during manufacturing, as a result of manufacturing, or after manufacturing as a result of a chemical reaction between the first inorganic salt containing calcium and the second inorganic salt containing phosphate.
6. The composition of statement 1, wherein the bioactive material is formed in situ during manufacturing, as a result of manufacturing, or after manufacturing.
7. The composition of statement 1, wherein the bioactive material is formed in situ during manufacturing, as a result of manufacturing, or after manufacturing and wherein the bioactive material is a blend of calcium phosphate salts including hydroxyapatite.
8. The composition of statement 1, wherein the bioactive material is formed in situ as a result of manufacturing and predominantly (>50%) consists of hydroxyapatite.
9. The composition of statement 1, wherein the bioactive material is formed in situ as a result of manufacturing and predominantly (>50%) consists of chlorine-substituted hydroxyapatite.
10. The composition of statement 1, wherein the bioactive material is formed in situ as a result of manufacturing and predominantly (>50%) consists of monetite.
11. The composition of statement 1, wherein the bioactive material is formed in situ as a result of manufacturing and predominantly (>50%) consists of brushite.
12. The composition of statement 1, wherein the bioactive material is a bioglass.
13. The composition of statement 1, wherein the composition contains free calcium ions at a concentration between 300 ppb and 5000 ppb.
14. The composition of statement 1, wherein the composition contains free phosphate ions at a concentration between 1 ppb and 500 ppb.
15. The composition of statement 1, wherein the composition contains free potassium ions at a concentration between 100 ppb and 10 ppm.
16. The composition of statement 1, wherein the composition contains free calcium and phosphate ions in equilibrium with suspended and stabilized hydroxyapatite.
17. The composition of statement 1, wherein the composition contains free calcium and phosphate ions in equilibrium with at least one apatite precursor and at least one apatite.
18. The composition of statement 1, wherein the bioactive material's particle size is less than one micron in size.
19. The composition of statement 1, wherein the buffering agent is either TRIS or HEPES.
20. The composition of statement 1, wherein the buffering agent is at a weight/weight concentration (w/w %) between 0.1%-1%.
21. The composition of statement 1, wherein the thickening agent is poloxamer 407.
22. The composition of statement 1, wherein the composition is substantially fluoride-free (i.e. <10 ppm fluoride).
23. The composition of statement 1, wherein the composition does not contain animal derived ingredients.
24. The composition of statement 1, further comprising at least one pH modifier.
25. The composition of statement 1, wherein the composition has a pH between 4 and 8.
26. The composition of statement 1, wherein the composition has a pH between 6 and 8.
27. The composition of statement 1, wherein the at least one surfactant comprises an anionic or nonionic surfactant.
28. The composition of statement 1, wherein the composition is isotonic (270-310 mOsm/L) to slightly hypertonic (310-1000 mOsm/L).
29. The composition of statement 1, wherein the remineralizing composition is used to treat sensitivity and remineralize dentin tissues in vivo.
30. The composition of statement 1, wherein the ratio between the first inorganic salt that yields free calcium (Ca²⁺) ions in water and the second inorganic salt yields free phosphate ions in water is between 1.5:1 and 2:1.
31. The composition of statement 1, wherein the at least two inorganic salts yield the following aqueous ion concentrations: sodium 120-160 mM, potassium 3-7 mM, magnesium 1-2 mM, calcium 2-5,000 mM, chloride 50-200 mM, bicarbonate 4-50 mM, phosphate 0.5-5,000 mM, and sulfate.
32. The composition of statement 1, wherein the at least two inorganic salts are at a weight/weight concentration (w/w %) between 0.01% and 2%.
33. The composition of statement 1, wherein the at least one bioactive material is formed in situ and is comprised of at least one apatite precursor, at least one apatite, and combinations thereof.
34. The composition of statement 1, wherein the at least one bioactive material is formed in situ and is comprised of at least one apatite precursor consisting of monetite or brushite.
35. The composition of statement 1, wherein the at least one bioactive material is formed in situ and is comprised of at least one apatite consisting of hydroxyapatite or chlorine-substituted hydroxyapatite.
36. The composition of statement 1, wherein the at least one bioactive material is formed in situ and is comprised of at least one apatite precursor consisting of monetite or brushite, and at least one apatite consisting of hydroxyapatite or chlorine-substituted hydroxyapatite.
37. The composition of statement 1, wherein the at least one bioactive material is formed in situ and is comprised of at least one apatite precursor and at least one apatite, wherein the ratio between the at least one apatite and the at least one apatite precursor is between 1:1 and 5:1.
38. The composition of statement 1, wherein the at least one bioactive material is formed in situ and is comprised of apatite precursors consists of at least 30% calcium phosphate salts.
39. The composition of statement 1, wherein the at least one bioactive material is formed in situ and is at a weight/weight concentration (w/w %) between 1% and 10%.
40. The composition of statement 1, wherein the remineralizing solution exhibits a surface tension less than 100 dynes/cm.
41. The composition of statement 1, further comprising at least one thickening agent.
42. The composition of statement 1, further comprising at least one thickening agent at a weight/weight concentration (w/w %) between 10%-25%.
43. The composition of statement 1, further comprising at least one thickening agent consisting of a poloxamer at a weight/weight concentration (w/w %) between 10%-25%.
44. The composition of statement 1, wherein the remineralizing solution exhibits a gel-like consistency.
45. The composition of statement 1, wherein the remineralizing solution exhibits a free-flowing liquid-like consistency.
46. The composition of statement 1, wherein the remineralizing solution exhibits a gel-like consistency with a viscosity between 500,000 mPa*s to 2,000,000 mPa*s.
47. The composition of statement 1, further comprising at least one antimicrobial agent.
48. The composition of statement 1, further comprising at least one antimicrobial agent wherein the at least one antimicrobial agent is selected from sodium benzoate, potassium sorbate, sodium methylparaben, sodium propylparaben, or combinations thereof.
49. The composition of statement 1, further comprising at least one antimicrobial agent at a concentration between 10 ppm and 2500 ppm.
50. The composition of statement 1, further comprising at least one antioxidant.
51. The composition of statement 1, further comprising at least one antioxidant between 0.25% and 1.0%.
52. The composition of statement 1, further comprising at least one inorganic salt that contains potassium.
53. The composition of statement 1, further comprising at least one inorganic salt that contains potassium, wherein the at least one inorganic salt that contains potassium is added at a weight/weight concentration (w/w %) between 0.04% and 0.1%.
54. The composition of statement 1, further comprising at least one antimicrobial agent consisting of hydrogen peroxide.
55. The composition of statement 1, further comprising at least one antimicrobial agent consisting of hydrogen peroxide at a weight/weight concentration (w/w %) between 0.1% and 2.5%.
56. The composition of statement 1, further comprising at least one collagen crosslinker consisting of glutaraldehyde.
57. The composition of statement 1, further comprising at least one collagen crosslinker consisting of glutaraldehyde at a weight/weight concentration (w/w %) between 0.05% and 3%.
58. A method of treating tooth sensitivity in vivo, comprising:
   introducing a remineralizing composition of any one of statement 1-57 onto a tooth surface or surfaces,
   maintaining contact of the remineralizing composition with the tooth surface for a designated period of time, and
   removal of the remineralizing composition from the tooth surface,
   wherein the remineralizing composition remineralizes hydroxyapatite present within the teeth.
59. The method of statement 58, wherein the designated period of time of contact of the composition with the tooth surface is between 30 seconds and 30 minutes.
60. The method of statement 58, wherein the remineralizing composition is applied in vivo using a custom dental tray or appliance.
61. The method of statement 58, wherein the remineralizing composition is packaged in a unit dose format and applied in vivo using an applicator brush contained therein.
62. The method of statement 58, wherein the remineralizing composition is applied in vivo using an applicator brush.
63. The method of statement 58, wherein the remineralizing composition occludes dentinal tubules in vivo.
64. The method of statement 58, wherein 1 mL to 30 mL of remineralizing composition is applied in vivo.
65. The method of statement 58, wherein the remineralizing of hydroxyapatite present within teeth occurs to a depth of 100 microns.
66. A method of treating incipient caries in vivo, comprising:
   introducing a remineralizing composition of any one of statement 1-57 onto a tooth surface or surfaces,
   maintaining contact of the remineralizing composition with the tooth surface for a designated period of time, and
   removal of the remineralizing composition from the tooth surface,
   wherein the remineralizing composition remineralizes hydroxyapatite present within the teeth.
67. A method of treating white spot lesions in vivo, comprising:
   introducing a remineralizing composition of any one of statement 1-57 onto a tooth surface or surfaces,
   maintaining contact of the remineralizing composition with the tooth surface for a designated period of time, and
   removal of the remineralizing composition from the tooth surface,
   wherein the remineralizing composition remineralizes hydroxyapatite present within the teeth.
68. A method of treating dentin and enamel demineralization in vivo, comprising:
   introducing a remineralizing composition of any one of statement 1-57 onto a tooth surface or surfaces,
   maintaining contact of the remineralizing composition with the tooth surface for a designated period of time, and
   removal of the remineralizing composition from the tooth surface,
   wherein the remineralizing composition remineralizes hydroxyapatite present within the teeth.
69. The method of statements 58, 66-68, wherein the remineralizing composition is applied in vivo following the application of a bioactive peptide.

What is claimed is:

1. A dental remineralizing composition, comprising:
at least two inorganic salts,
at least one bioactive material,
at least one buffering agent,
at least one solvent, and,
free potassium ions at a concentration between 100 ppb and 10 ppm;
wherein at least a first inorganic salt yields free calcium (Ca²⁺) ions in water,
wherein at least a second inorganic salt yields free phosphate ions in water;
wherein the bioactive material is formed in situ during manufacturing;
wherein the composition contains free calcium and phosphate ions in equilibrium with suspended and stabilized hydroxyapatite;
wherein the remineralizing solution exhibits a gel-like consistency.

2. The composition of claim 1, further comprising one or more of a thickener, a flavorant, a sweetener, a surfactant, a hydrotrope, a colorant, an antimicrobial agent, a preservative, a prebiotic, a probiotic, an antioxidant, a chelator, or a sequestrant.

3. The composition of claim 1, wherein the bioactive material's particle size is less than one micron in size.

4. The composition of claim 1, wherein the buffering agent is either TRIS or HEPES.

5. The composition of claim 1, wherein the thickening agent is poloxamer 407.

6. The composition of claim 1, wherein the composition has less than about 10 ppm fluoride.

7. The composition of claim 1, wherein the composition has a pH between 4 and 8.

8. The composition of claim 1, wherein the at least one bioactive material is formed in situ and is comprised of at least one apatite precursor, at least one apatite, and combinations thereof.

9. The composition of claim 1, wherein the ratio between the first inorganic salt that yields free calcium (Ca²⁺) ions in water, and the second inorganic salt yields free phosphate ions in water is between 1.5:1 and 2:1.

10. The composition of claim 1, wherein the at least one bioactive material is formed in situ and is at a weight/weight concentration (w/w %) between 1% and 10%.

11. The composition of claim 1, further comprising at least one antimicrobial agent.

12. The composition of claim 1, further comprising at least one antimicrobial agent wherein the at least one antimicrobial agent is selected from sodium benzoate, potassium sorbate, sodium methylparaben, sodium propylparaben, or combinations thereof.

13. The composition of claim 1, further comprising at least one antioxidant.

14. The composition of claim 1, further comprising at least one antimicrobial agent consisting of hydrogen peroxide at a weight/weight concentration (w/w %) between 0.1% and 2.5%.

15. A method of treating tooth sensitivity in vivo, comprising:
introducing a remineralizing composition of claim 1 onto a tooth surface or surfaces,
maintaining contact of the remineralizing composition with the tooth surface for a designated period of time, and
removal of the remineralizing composition from the tooth surface,
wherein the remineralizing composition remineralizes hydroxyapatite present within the teeth.

16. The method of claim 15, wherein the designated period of time of contact of the composition with the tooth surface is between 30 seconds and 30 minutes.

17. The method of claim 15, wherein the remineralizing composition is applied in vivo using a custom dental tray or appliance.

18. The method of claim 15, wherein the remineralizing composition occludes dentinal tubules in vivo.

19. The method of claim 15, wherein the remineralizing of hydroxyapatite present within teeth occurs to a depth of 100 microns.

20. The method of claim 15, wherein the remineralizing composition is applied in vivo following the application of a bioactive peptide.
